(19) United States
(12) Patent Application Publication
(10) Pub. No.: US 2022/0211815 A1
(43) Pub. Date: Jul. 7, 2022

(54) COMBINATION THERAPY FOR THE TREATMENT OF CANCER

(71) Applicants: Novartis AG, Basel (CH); Chinook Therapeutics, Inc., Seattle, WA (US)

(72) Inventors: Nadja KOPP, Cambridge, MA (US); Justin LEONG, Berkeley, CA (US); Jeffrey MCKENNA, Carlisle, MA (US); Chudi Obioma NDUBAKU, Berkeley, CA (US); Maria PINZON-ORTIZ, Upton, MA (US); Xianhui RONG, Boxborough, MA (US); Ryan SULLIVAN, Watertown, MA (US)

(21) Appl. No.: 16/966,514

(22) PCT Filed: Feb. 1, 2019
(86) PCT No.: PCT/IB2019/050806

§ 371 Date: Jul. 31, 2020

Related U.S. Application Data
(60) Provisional application No. 62/625,671, filed on Feb. 2, 2018.

(51) Int. Cl. A61K 38/20; A61K 38/17; A61K 45/06; A61P 35/00

(52) U.S. Cl. CPC A61K 38/2086; A61P 35/00; A61K 45/06; A61K 38/1793

(57) ABSTRACT

The present disclosure relates to a pharmaceutical composition comprising a STING agonist molecule in combination with an IL-15/IL-15Ra complex. The present combination can be administered independently or separately, in a quantity which is therapeutically effective for the treatment of cancer. Also provided is the use of such a combination for the manufacture of a medicament; the use of such a combination as a medicine; a kit of parts comprising such a combination; and a method of treatment of such a combination.

### FIELD

The present disclosure relates to STING agonist molecules in combination with an additional agent that enhances their efficacy, such as a complex comprising interleukin-15 (“IL-15”) bound to IL-15 receptor alpha (“IL-15Ra”). In a specific aspect, the combination is useful in the prevention, treatment, and/or management of disorders in which inducing innate immunity is beneficial, such as cancer.

### SEQUENCE LISTING

The instant application contains a Sequence Listing which has been submitted electronically in ASCII format and is hereby incorporated by reference in its entirety. Said ASCII copy is named PAT058052-US-PCT_SL.txt and is 33,923 bytes in size.

### BACKGROUND

Innate immunity is a rapid nonspecific immune response that fights against environmental insults including, but not limited to, pathogens such as bacteria or viruses. Adaptive immunity is a slower but more specific immune response, which confers long-lasting or protective immunity to the host and involves differentiation and activation of naive T lymphocytes into CD4+ T helper cells and/or CD8+ cytotoxic T cells, to promote cellular and humoral immunity. Antigen presentation cells of the innate immune system, such as dendritic cells or macrophages, serve as a critical link between the innate and adaptive immune systems by phagocytosing and processing the foreign antigens and presenting them on the cell surface to the T cells, thereby activating T cell response.

STING (stimulator of interferon genes) is an endoplasmic reticulum adaptor that facilitates innate immune signaling (Ishikawa and Barber, Nature 2008, 455(7213):674-678). It was reported that STING comprises four putative transmembrane regions (Ouyang et al., Immunity (2012) 36, 1073), predominantly resides in the endoplasmic reticulum and is able to activate NF-kB, STAT6, and IRF3 transcription pathways to induce expression of type I interferon (e.g., IFN-α and IFN-β) and exert a potent anti-viral state following expression (Ishikawa and Barber, Nature 2008, 455(7213):674-678; Chen et al., Cell (2011) 147, 436-446). In contrast, loss of STING rendered murine embryonic fibroblasts extremely susceptible to negative stranded virus infection, including vesicular stomatitis virus. (Ishikawa and Barber, Nature. 2008, 455(7213):674-678).

The cytokine, interleukin-15 (IL-15), is a member of the four alpha-helix bundle family of lymphokines produced by many cells in the body. IL-15 plays a pivotal role in modulating the activity of both the innate and adaptive immune system, e.g., maintenance of the memory T-cell response to invading pathogens, inhibition of apoptosis, activation of dendritic cells, and induction of Natural Killer (NK) cell proliferation and cytotoxic activity. IL-15 signaling has been shown to occur through the heterodimeric complex of the IL-15 receptor, which consists of three polypeptides, the type-specific IL-15 receptor alpha (“IL-15Ra”), the IL-2/IL-15 receptor beta (or CD122) (“β”), and the common gamma chain (or CD132) (“γ”) that is shared by multiple cytokine receptors. Based on its multifaceted role in the immune system, various therapies designed to modulate IL-15-mediated function have been explored. Recent reports suggest that IL-15, when complexed with the sIL-15Ra, or the sushi domain, maintains its immune enhancing function. Recombinant IL-15 and IL-15/IL-15Ra complexes have been shown to promote to different degrees the expansion of memory CD8 T cells and NK cells and enhance tumor rejection in various preclinical models. Furthermore, tumor targeting of IL-15 or IL-15/IL-15Ra complex containing constructs in mouse models, resulted in improved anti-tumor responses in either immunocompetent animals transplanted with syngeneic tumors or in T- and B cell-deficient SCID mice (retaining NK cells) injected with human tumor cell lines. Enhanced anti-tumor activity is thought to be dependent on increased half-life of the IL-15-containing moiety as well as the trans-presentation of IL-15 on the surface of tumor cells leading to enhanced NK and/or CD8 cytotoxic T cell expansion within the tumor. As such, tumor cells engineered to express IL-15 were also reported to promote rejection of established tumors by enhancing T cell and NK cell recruitment, proliferation and function (Zhang et al., (2009) PNAS USA. 106:7513-7518; Munger et al., (1995) Cell Immunol. 165(2):289-293; Evans et al., (1997) Cell Immunol. 179(1):66-73; Klebanoff et al., (2004) PNAS USA. 101(7):1969-74; Sneller et al., (2011) Blood.118(26):6845-6848; Zhang et al., (2012) J. Immunol. 188(12):6156-6164).

Therefore, therapeutic approaches that enhance anti-tumor immunity could work more effectively when the immune response is optimized by targeting multiple components at one or more stages of an immune response. Therefore there remains an unmet need for new immunotherapies for the treatment of disease, in particular cancer.

### SUMMARY

Accordingly, disclosed herein are combination therapies that enhance anti-tumor immunity and as such can provide a superior beneficial effect, e.g., in the treatment of a disorder, such as an enhanced anti-cancer effect, reduced toxicity and/or reduced side effects, compared to monotherapy administration of the therapeutic agents of the combination. For example, one or more of the therapeutic agents in the combination can be administered at a lower dosage, or for a shorter period of administration or less frequently, than would be required to achieve the same therapeutic effect compared to the monotherapy administration. More specifically, one of the therapeutic agents in the combination can be administered to enhance the effect of the other agent. Therefore, compositions and methods for treating cancer using combination therapies are disclosed.

In an embodiment, the present disclosure provides a combination comprising a STING agonist molecule in combination with an IL-15/IL-15Ra complex.

In one embodiment, STING agonist molecule, is a dinucleotide. In another embodiment the STING agonist molecule is a cyclic dinucleotide (CDN). In embodiments disclosed herein, the STING agonist molecule is selected from the group consisting of:

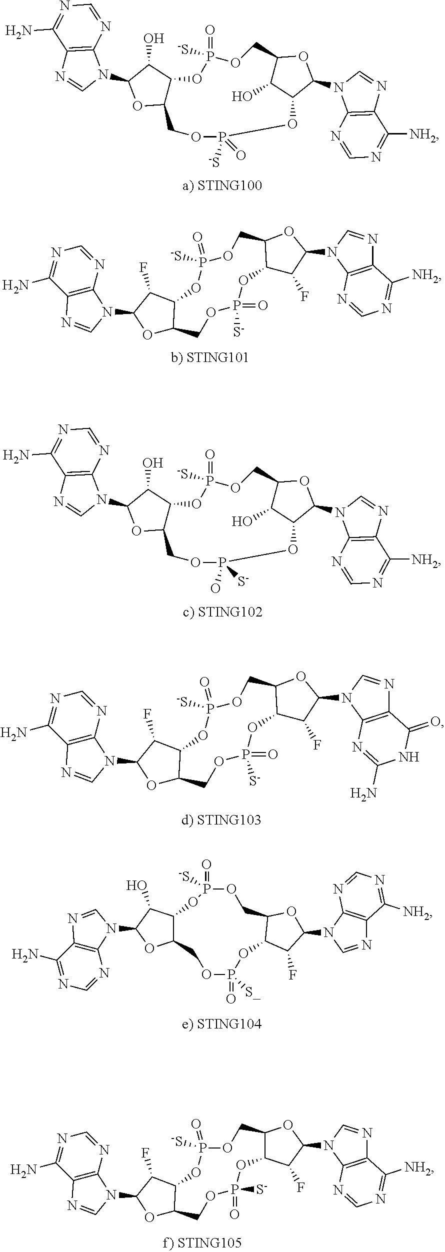

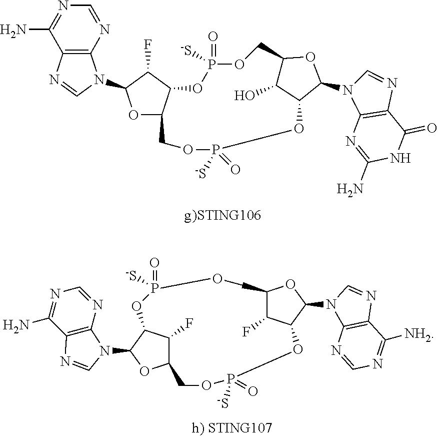

In one embodiment, the IL-15/IL-15Ra complex of the combination can comprise wild type IL-15 or an IL-15 derivative covalently or noncovalently bound to wild type IL-15Ra or an IL-15Ra derivative. In one embodiment, the IL-15/IL-15Ra complex comprises wild type IL-15 and wild type IL-15Ra. In another embodiment, the IL-15/IL-15Ra complex comprises an IL-15 derivative and wild type IL-15Ra. In another embodiment, the IL-15/IL-15Ra complex is in the wild type heterodimeric form. In another embodiment, the IL-15 is human IL-15 and IL-15Ra is human IL-15Ra. In a specific embodiment, the human IL-15 comprises the amino acid sequence of SEQ ID NO: 1 or amino acid residues 49 to 162 of SEQ ID NO: 1 and the human IL-15Ra comprises the amino acid sequence of SEQ ID NO: 6 or a fragment thereof, as described in Table 1. In another embodiment the IL-15 comprises the amino acid sequence of SEQ ID NO: 1 or amino acid residues 49 to 162 of SEQ ID NO: 1 and the IL-15Ra comprises the amino acid sequence of SEQ ID NO: 7 or SEQ ID NO: 10, as described in Table 1. In specific embodiments, the human IL-15 comprises amino acid residues 49 to 162 of the amino acid sequence of SEQ ID NO: 1 and human IL-15Ra comprises the amino acid sequence of SEQ ID NO: 10, as described in Table 1.

In other embodiments, the IL-15Ra is glycosylated such that glycosylation accounts for at least or more than 20%, 30%, 40% or 50% of the mass of the IL-15Ra. In another embodiment, the IL-15/IL-15Ra complex comprises wild type IL-15 and an IL-15Ra derivative. In another embodiment, the IL-15/IL-15Ra complex comprises an IL-15 derivative and an IL-15Ra derivative. In one embodiment, the IL-15Ra derivative is a soluble form of the wild type IL-15Ra. In another embodiment, the IL-15Ra derivative comprises a mutation that inhibits cleavage by an endogenous protease. In a specific embodiment, the extracellular domain cleavage site of IL-15Ra is replaced with a cleavage site that is specifically recognized by a heterologous protease. In one embodiment, the extracellular domain cleavage site of IL-15Ra is replaced with a heterologous extracellular domain cleavage site (e.g., heterologous transmembrane domain that is recognized and cleaved by another enzyme unrelated to the endogenous processing enzyme that cleaves the IL-15Ra).

In a specific embodiment, the present disclosure provides a combination comprising a STING agonist molecule in combination with an IL-15/IL-15Ra complex, wherein i) the STING agonist molecule is selected from the group consisting of the molecules STING100-STING107; ii) the IL-15/IL-15Ra complex is a heterodimeric complex of human IL-15 and human soluble IL-15Ra and wherein the human IL-15 and comprises residues 49 to 162 of the amino acid sequence of SEQ ID NO: 1 and the human soluble IL-15Ra comprises the amino acid sequence of SEQ ID NO: 10.

### Uses of the Combination Therapies

The combinations disclosed herein can result in one or more of: anti-tumor immunity, an increase in immune cell function (e.g., one or more of CD8+ T cell proliferation, NK cell proliferation, inhibition of regulatory T cell function, an effect on the activity of multiple cell types, such as CD8+ T cells and NK cells), and an increase in tumor infiltrating lymphocytes. In one embodiment, the use of an IL-15/IL-15Ra complex in the combination stimulates the immune response and can enhance innate immunity resulting from the use of a STING agonist molecule. Thus, such combinations can be used to treat or prevent disorders where enhancing anti-tumor immunity in a subject is desired, e.g. cancer. Such combination therapies can be used, e.g., for cancer immunotherapy and treatment of other conditions, such as chronic infection. In an embodiment, provided herein are methods of treating (e.g., inhibiting, reducing, ameliorating, or preventing) a disorder, e.g., a hyperproliferative condition or disorder (e.g., a cancer) in a subject by administering to the subject a STING agonist molecule in combination with an IL-15/IL-15Ra complex. Also provided is a STING agonist molecule in combination with an IL-15/IL-15Ra complex for use in the treatment of (e.g., inhibiting, reducing, ameliorating, or preventing) a disorder, e.g., a hyperproliferative condition or disorder (e.g., a cancer) in a subject. Further provided is a STING agonist molecule in combination with an IL-15/IL-15Ra complex for use in the preparation of a medicament for the treatment of (e.g., inhibiting, reducing, ameliorating, or preventing) a disorder, e.g., a hyperproliferative condition or disorder (e.g., a cancer) in a subject.

The augmentation of anti-tumor immunity of a STING agonist molecule and an IL-15/IL-15Ra complex has been demonstrated in Example 1 as described below. In some embodiments, the combination can be used to treat cancer. Cancer includes, but is not limited to; sarcomas, adenocarcinomas, blastomas, and carcinomas, of the various organ systems, such as those affecting liver, lung, breast, lymphoid, biliarintestinal (e.g., colon), genitourinary tract (e.g., renal, urothelial cells), prostate and pharynx.

Adenocarcinomas include malignancies such as most colon cancers, rectal cancer, renal-cell carcinoma, liver cancer, small cell lung cancer, non-small cell carcinoma of the lung, cancer of the small intestine and cancer of the esophagus. In one embodiment, the cancer is a melanoma, e.g., an advanced stage melanoma. Examples of other cancers that can be treated include bone cancer, pancreatic cancer, skin cancer, cancer of the head or neck, cutaneous or intraocular malignant melanoma, uterine cancer, ovarian cancer, rectal cancer, colorectal cancer, cancer of the peritoneum, stomach or gastric cancer, esophageal cancer, salivary gland carcinoma, testicular cancer, uterine cancer, carcinoma of the fallopian tubes, carcinoma of the endometrium, carcinoma of the cervix, carcinoma of the vagina, carcinoma of the vulva, penile carcinoma, glioblastoma, neuroblastoma, cervical cancer, Hodgkin Disease, non-Hodgkin lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, neuroendocrine tumors (including carcinoid tumors, gastrinoma, and islet cell cancer), mesothelioma, schwannoma (including acoustic neuroma), meningioma, epidermoid cancer, squamous cell cancer, T-cell lymphoma, B-cell acute lymphoid leukemia (“BALL”), T-cell acute lymphoid leukemia (“TALL”), acute lymphoid leukemia (ALL); one or more chronic leukemias including but not limited to, e.g., chronic myelogenous leukemia (CML), chronic lymphocytic leukemia (CLL); additional hematologic cancers or hematologic conditions including, but not limited to, e.g., B cell prolymphocytic leukemia, blastic plasmacytoid dendritic cell neoplasm, Burkitt's lymphoma, diffuse large B cell lymphoma, Follicular lymphoma, Hairy cell leukemia, small cell- or a large cell-follicular lymphoma, malignant lymphoproliferative conditions, MALT lymphoma, mantle cell lymphoma, Marginal zone lymphoma, multiple myeloma, myelodysplasia and myelodysplastic syndrome, non-Hodgkin lymphoma, plasmablastic lymphoma and plasmacytoid dendritic cell neoplasm.

In one embodiment, the combination of a STING agonist molecule and IL-15/IL-15Ra complex are administered to a subject separately or together. In another embodiment, the combination of a STING agonist molecule and IL-15/IL-15Ra complex are administered simultaneously or sequentially.

The present application also provides nucleic acids encoding the IL-15/IL-15Ra complex disclosed herein, as well as a vector comprising the nucleic acid, and a host cell comprising the nucleic acid or the vector. Also provided are methods of producing the IL-15/IL-15Ra complex disclosed herein, the method comprising: culturing a host cell expressing a nucleic acid encoding the IL-15/IL-15Ra complex; and collecting the IL-15/IL-15Ra complex from the culture.

### BRIEF DESCRIPTION OF THE FIGURES

FIG. 1 is a table showing the pharmacodynamics (PD) and efficacy of the combination of hetIL15 and a STING agonist molecule in a mouse model of colorectal cancer (MC38).

FIG. 2 shows the study design and dosing schedule of the combination in the mouse model of colorectal cancer.

FIG. 3A/B-FIG. 3A shows curves of the tumor volume of the mice in the colorectal cancer model with hetIL15 as a single therapeutic, STING100 as a single therapeutic and the hetIL15/STING100 combination with increasing doses of STING100. FIG. 3B is a graph of the survival of the mice with the same dosing regimen.

FIG. 4 is a graph depicting the body weight of the mice in the colorectal tumor model, demonstrating that hetIL15 and STING100 are well tolerated as single therapeutics as well as in combination.

FIG. 5A/F shows that STING100 or hetIL15 as monotherapy leads to only a modest tumor growth delay, but that the combination of STING100 and hetIL15 leads to durable complete responses. FIG. 5F indicates the complete recovery of 5 out of 7 mice in the cohort, a complete recovery rate of 71%.

FIG. 6A/C shows that the hetIL15/STING100 combination enhances anti-tumor immunity. FIG. 6A is a graph depicting the increase in CD8+ T cells, while FIG. 6B shows the increase in NK cells. FIG. 6C indicates the numbers of CD8+ T cells with hetIL15 and STING100 as single therapies, and then the hetIL15/STING100 combination with increasing doses of STING100.

FIG. 7A/B shows that mice treated with the hetIL15/STING100 combination develop durable anti-tumor immunity. FIG. 7A indicates that mice treated with the hetIL15/STING100 combination do not form tumors when re-challenged with the same type of tumor cells (MC38). FIG. 7B depicts the amount of IFNγ produced by splenocytes taken from normal, untreated mice, splenocytes taken from mice engrafted with MC38 colorectal tumors and mice treated with the hetIL15/STING100 combination and rechallenged.

### DETAILED DESCRIPTION

The present disclosure provides for a combination comprising a STING agonist molecule with an IL-15/IL-15Ra complex, and pharmaceutical compositions, production methods, and methods of use of such a combination.

### Definitions

Unless defined otherwise, all technical and scientific terms used herein have the same meaning as commonly understood by those of ordinary skill in the art to which this disclosure pertains.

The term “a STING agonist molecule,” as used herein, refers to a compound capable of binding to STING and activating STING. Activation of STING activity can include, for example, stimulation of inflammatory cytokines, including interferons, such as type 1 interferons, including IFN-α IFN-β, type 3 interferons, e.g., IFNγ, or other proinflammatory molecules including but not limited to IP10, TNF, IL-6, CXCL9, CXCL10, CCL4, CXCL11, CCL5, CCL3, or CCL8. STING agonist activity can also include stimulation of TANK binding kinase (TBK) 1 phosphorylation, STING phosphorylation, interferon regulatory factor (IRF) activation (e.g., IRF3 activation), NFκB activation, STAT6 activation, secretion of interferon-γ-inducible protein (IP-10), or other inflammatory proteins and cytokines. STING agonist activity can be determined, for example, by the ability of a compound to stimulate activation of the STING pathway as detected using an interferon stimulation assay, a reporter gene assay (e.g., a hSTING wt assay, or a THP-1 Dual assay), a TBK1 activation assay, IP-10 assay, a STING Biochemical [3H]cGAMP Competition Assay, or other assays known to persons skilled in the art. STING agonist activity can also be determined by the ability of a compound to increase the level of transcription of genes that encode proteins activated by STING or the STING pathway. Such activity can be detected, for example, quantitative real time PCR, RNAseq, Nanostring or various assays for detection of secreted proteins (cytokine bead array, ELISA). In some embodiments, an assay to test for activity of a compound in a STING knock-out cell line can be used to determine if the compound is specific for STING, wherein a compound that is specific for STING would not be expected to have activity in a cell line wherein the STING pathway is partially or wholly deleted.

By “in combination with” or “ a combination of,” it is not intended to imply that the agents of the combination must be administered at the same time and/or formulated for delivery together, although these methods of delivery are within the scope described herein. The STING agonist molecule can be administered prior to, concurrently with, or subsequent to, the IL-15/IL-15Ra complex and vice versa, i.e. the STING agonist molecule and the IL-15/IL-15Ra complex can be administered in any order. In general, each agent will be administered at a dose and/or on a time schedule determined for that agent. It will further be appreciated that each therapeutic agent utilized in this combination can be administered together in a single composition or administered separately in different compositions. In general, it is expected that the therapeutic agents of the combination be utilized at levels that do not exceed the levels at which they are utilized individually. In some embodiments, the levels of the agents utilized in combination will be lower than those utilized individually. In some embodiments, the agents of the combination can also be used as entirely separate pharmaceutical dosage forms or pharmaceutical formulations that are also sold independently of each other and where instructions of the possibility of their combined use is or are provided in the package equipment, e.g. leaflet or the like, or in other information

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, gamma-carboxy glutamate, and O-phosphoserine. Amino acid analogs refer to compounds that have the same basic chemical structure as a naturally occurring amino acid, i.e., an alpha carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid Amino acid mimetics refers to chemical compounds that have a structure that is different from the general chemical structure of an amino acid, but that functions in a manner similar to a naturally occurring amino acid.

The term “conservatively modified variant” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

For polypeptide sequences, “conservatively modified variants” include individual substitutions, deletions or additions to a polypeptide sequence which result in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles. The following eight groups contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)). In one embodiment, the term “conservative sequence modifications” are used to refer to amino acid modifications that do not significantly affect or alter the binding characteristics of the IL-15/IL-15Ra complex containing the amino acid sequence.

The terms “identical” or percent “identity,” in the context of two or more nucleic acids or polypeptide sequences, refer to two or more sequences or subsequences that are the same. Two sequences are “substantially identical” if two sequences have a specified percentage of amino acid residues or nucleotides that are the same (i.e., 60% identity, optionally 65%, 70%, 75%, 80%, 85%, 90%, 95%, 96%, 97%, 98% or 99% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 50 nucleotides (or 10 amino acids) in length, or more preferably over a region that is 100 to 500 or 1000 or more nucleotides (or 20, 50, 200 or more amino acids) in length. Optionally, the identity exists over a region that is at least 50 nucleotides (or 10 amino acids) in length, or more preferably over a region that is 100 to 500 or 1000 or more nucleotides (or 20, 50, 200 or more amino acids) in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to 600, usually about 50 to about 200, more usually about 100 to about 150 in which a sequence can be compared to a reference sequence of the same number of contiguous positions after the two sequences are optimally aligned. Methods of alignment of sequences for comparison are well known in the art. Optimal alignment of sequences for comparison can be conducted, e.g., by the local homology algorithm of Smith & Waterman (1970) Adv. Appl. Math. 2:482c, by the homology alignment algorithm of Needleman & Wunsch, (1970) J. Mol. Biol. 48:443, by the search for similarity method of Pearson & Lipman, (1988) PNAS USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Brent et al., Current Protocols in Molecular Biology, John Wiley & Sons, Inc. (ringbou ed., 2003)).

Two examples of algorithms that are suitable for determining percent sequence identity and sequence similarity are the BLAST and BLAST 2.0 algorithms, which are described in Altschul et al., (1977) Nuc. Acids Res. 25:3389-3402; and Altschul et al., (1990) J. Mol. Biol. 215:403-410, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (N) of 11, an expectation (E) or 10, M=5, N=-4 and a comparison of both strands. For amino acid sequences, the BLASTP program uses as defaults a word length of 3, and expectation (E) of 10, and the BLOSUM62 scoring matrix (see Henikoff & Henikoff, (1989) PNAS USA 89:10915) alignments (B) of 50, expectation (E) of 10, M=5, N=-4, and a comparison of both strands.

The BLAST algorithm also performs a statistical analysis of the similarity between two sequences (see, e.g., Karlin & Altschul, (1993) PNAS USA 90:5873-5787). One measure of similarity provided by the BLAST algorithm is the smallest sum probability (P (N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The percent identity between two amino acid sequences can also be determined using the algorithm of E. Meyers and W. Miller (Comput. Appl. Biosci., (1988) 4:11-17) which has been incorporated into the ALIGN program (version 2.0), using a PAM120 weight residue table, a gap length penalty of 12 and a gap penalty of 4. In addition, the percent identity between two amino acid sequences can be determined using the Needleman & Wunsch (J. Mol, Biol.(1970) 48:444-453) algorithm which has been incorporated into the GAP program in the GCG software package, using either a BLOSUM62 matrix or a PAM250 matrix, and a gap weight of 16, 14, 12, 10, 8, 6, or 4 and a length weight of 1, 2, 3, 4, 5, or 6.

Other than percentage of sequence identity noted above, another indication that two nucleic acid sequences or polypeptides are substantially identical is that the polypeptide encoded by the first nucleic acid is immunologically cross-reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The term “nucleic acid” is used herein interchangeably with the term “polynucleotide” and refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, as detailed below, degenerate codon substitutions can be achieved by generating sequences in which the third position of one or more selected (or all) codons is substituted with mixed-base and/or deoxyinosine residues (Batzer et al., (1991) Nucleic Acid Res. 19:5081; Ohtsuka et al., (1985) J. Biol. Chem. 260:2605-2608; and Rossolini et al., (1994) Mol. Cell. Probes 8:91-98).

The term “operably linked” refers to a functional relationship between two or more polynucleotide (e.g., DNA) segments. Typically, it refers to the functional relationship of a transcriptional regulatory sequence to a transcribed sequence. For example, a promoter or enhancer sequence is operably linked to a coding sequence if it stimulates or modulates the transcription of the coding sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance

As used herein, the term, “optimized” means that a nucleotide sequence has been altered to encode an amino acid sequence using codons that are preferred in the production cell or organism, generally a eukaryotic cell, for example, a cell of Pichia, a Chinese Hamster Ovary cell (CHO) or a human cell. The optimized nucleotide sequence is engineered to retain completely or as much as possible the amino acid sequence originally encoded by the starting nucleotide sequence, which is also known as the “parental” sequence. The optimized sequences herein have been engineered to have codons that are preferred in mammalian cells. However, optimized expression of these sequences in other eukaryotic cells or prokaryotic cells is also envisioned herein. The amino acid sequences encoded by optimized nucleotide sequences are also referred to as optimized.

The terms “polypeptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. Unless otherwise indicated, a particular polypeptide sequence also implicitly encompasses conservatively modified variants thereof.

The term “recombinant host cell” (or simply “host cell”) refers to a cell into which a recombinant expression vector has been introduced. It should be understood that such terms are intended to refer not only to the particular subject cell but to the progeny of such a cell. Because certain modifications can occur in succeeding generations due to either mutation or environmental influences, such progeny may not, in fact, be identical to the parent cell, but are still included within the scope of the term “host cell” as used herein.

The term “subject” includes human and non-human animals. Non-human animals include all vertebrates, e.g., mammals and non-mammals, such as non-human primates, sheep, dog, cow, chickens, amphibians, and reptiles. Except when noted, the terms “patient” or “subject” are used herein interchangeably.

The terms “treat,” “treated,” “treating,” and “treatment,” include the administration of compositions to alleviate or delay the onset of the symptoms, complications, or biochemical indicia of a disease, preventing the development of further symptoms or arresting or inhibiting further development of the disease, condition, or disorder. Treatment can be measured by the therapeutic measures described hererin. The methods of “treatment” include administration of STING agonist molecule in combination with an IL-15/IL-15Ra complex to a subject in order to cure, reduce the severity of, or ameliorate one or more symptoms of cancer or condition associated with cancer, in order to prolong the health or survival of a subject beyond that expected in the absence of such treatment. For example, “treatment” includes the alleviation of a disease symptom in a subject by at least 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95% or more.

The term “prevent” includes administration of compositions or combinations of STING agonist molecules and a IL-15/IL-15Ra complex to prevent or delay the onset of the disease, or to prevent the manifestation of clinical or subclinical symptoms thereof i.e. prophylactic administration, or therapeutic suppression or alleviation of symptoms after the manifestation of the disease.

The term “vector” is intended to refer to a polynucleotide molecule capable of transporting another polynucleotide to which it has been linked. One type of vector is a “plasmid,” which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” can be used interchangeably as the plasmid is the most commonly used form of vector. However, it is intended to include such other forms of expression vectors, such as viral vectors (e.g., replication defective retroviruses, adenoviruses and adeno-associated viruses), which serve equivalent functions.

### STING Agonist Molecules

Example synthesis of STING agonist molecules can be found according to the synthetic description in WO2014189805.

### Specifically, Compound (STING100),

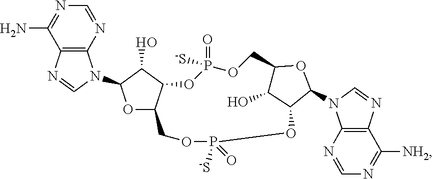

was synthesized according to the scheme below:

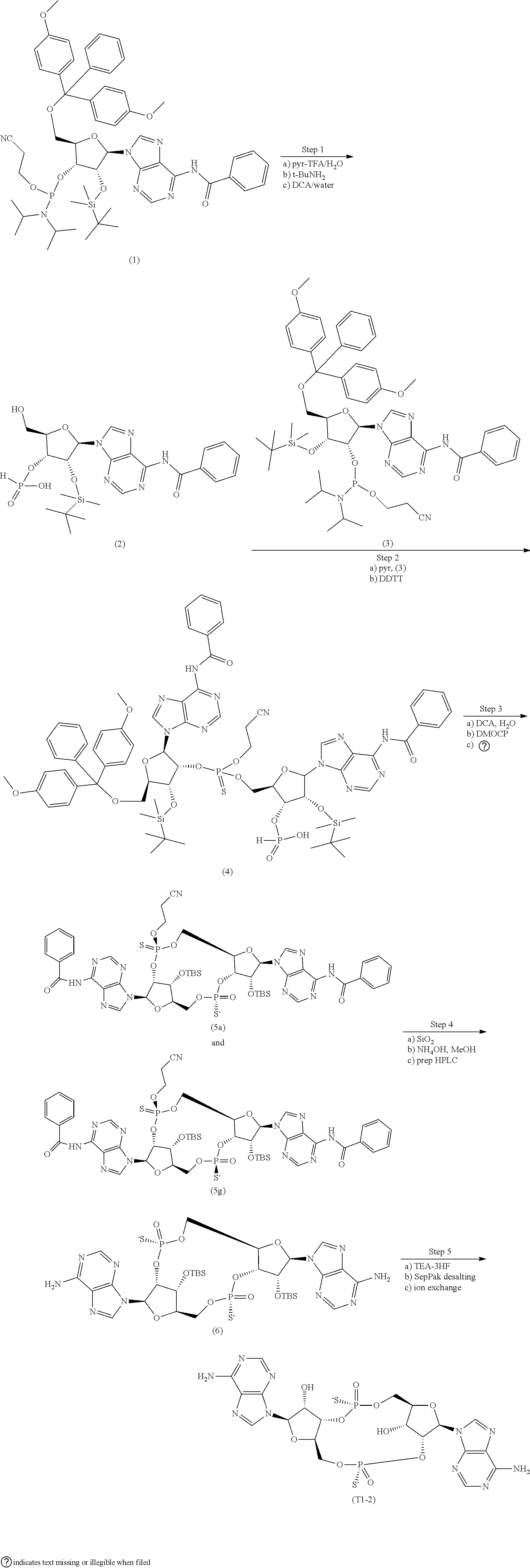

To a solution of 5 g (5.15 mmol) N -benzoyl-5′-O-(4, 4′-dimethoxytrityl)-2′-O-tert-butyldimethylsilyl-3′-O-[(2-cyanoethyl)-N, N-diisopropylaminophinyl]adenosine (1) in 25 ml acetonitrile was added 0.18 ml (10 mmole) water and 1.20 g (6.2 mmol) pyridinium trifluoroacetate. After 5 minutes stirring at room temperature 25 ml tertbutylamine was added and the reaction stirred for 15 minutes at room temperature. The solvents were removed under reduced pressure to give (2R,3R,4R,5R)-5-(6-benzamido-9H-purin-9-yl)-2-((bis(4-methoxyphenyl)(phenyl)methoxy)methyl)-4-((tert-butyldimethylsily)oxy)tetrahydrofuran-3-yl hydrogen phosphonate as a foam which was then coevaporated with acetonitrile (2×50 ml), then dissolved in 60 ml dichloromethane. To this solution was added water (0.9 ml, 50 mmole) and 60 ml of 6% (v/v) dichloroacetic acid (44 mmol) in dichloromethane. After 10 minutes at room temperature the reaction was quenched by the addition of pyridine (7.0 ml, 87 mmol), and concentrated to an oil which was dried by three co-evaporations with 40 ml anhydrous acetonitrile giving (2) in a volume of 12 ml.

N -benzoyl-5′-O-(4, 4′-dimethoxytrityl)-3′-O-tert-butyldimethylsilyl-2′-O-[(2-cyanoethyl)-N, N-diisopropylaminophinyl]adenosine ((3), 6.4 g, 6 6 mmole) was dissolved in 40 ml anhydrous acetonitrile and dried by three co-evaporations with 40 ml anhydrous acetonitrile, the last time leaving 20 ml. 3 Å molecular sieves were added and the solution stored under argon until used. Azeo dried (3) (6.4 g, 6 6 mmole) in 20 ml acetonitrile was added via syringe to a solution of (2) (5.15 mmol) in 12 ml of anhydrous acetonitrile. After 5 minutes stirring at room temperature, 1.14 g (5.6 mmol) of 3-((N,N-dimethylaminomethylidene)amino)-3H-1, 2,4-dithiazole-5-thione (DDTT) was added and the reaction stirred for 30 minutes at room temperature. The reaction was concentrated and the residual oil dissolved in 80 ml dichloromethane. Water (0.9 ml, 50 mmol) and 80 ml of 6% (v/v) dichloroacetic acid (58 mmol) in dichloromethane was added, and the reaction stirred for 10 minutes at room temperature. 50 ml pyridine was added to quench the dichloroacetic acid. The solvents were removed under reduced pressure to give crude (2R,3R,4R,5R)-5-(6-benzamido-9H-purin-9-yl)-2-((((((2R,3R,4R,5R)-2-(6-benzamido-9H-purin-9-yl)-4-((tert-butyldimethylsily)oxy)-5-(hydroxymethyl)tetrahydrofuran-3-ypoxy)(2-cyanoethoxy)phosphorothioyl)oxy)methyl)-4-((tert-butyldimethylsilyl)oxy)tetrahydrofuran-3-yl hydrogen phosphonate as a solid, which was then dissolved in 150 ml dry pyridine and concentrated down to a volume of approximately 100 ml. 2-chloro-5,5-dimethyl-1,3,2-dioxaphosphorinane-2-oxide (DMOCP, 3.44 g, 18 mmole) was then added and the reaction stirred for 5 minutes at room temperature. 3.2 ml water was added immediately followed by addition of 3-H-1,2-benzodithiol-3-one (1.3 g, 7.7 mmole), and the reaction stirred for 5 minutes at room temperature. The reaction mix was then poured into 700 ml water containing 20 g NaHCO₃ and stirred for 5 minutes at room temperature, then poured into a separatory funnel and extracted with 800 ml 1: lethyl acetate:diethyl ether. The aqueous layer was extracted again with 600 ml 1:1 ethyl acetate:diethyl ether. The organic layers were combined and concentrated under reduced pressure to yield approximately 11 g of an oil containing diastereoisomers (5a) and (5b). The crude mixture above was dissolved in dichloromethane and applied to a 250 g silica column. The desired diastereoisomers were eluted from the column using a gradient of ethanol in dichloromethane (0-10%). Fractions containing the desired diastereoisomers (5a) and (5b) were combined and concentrated, giving 2.26 g of approximately 50% (5a) and 50% (5b).

2.26 g of crude (5a) and (5b) from the silica gel column was transferred to a thick-walled glass pressure tube. 60 ml methanol and 60 ml concentrated aqueous ammonia was added and the tube was heated with stirring in an oil bath at 50° C. for 16 h. The reaction mixture was cooled to near ambient temperature, sparged with a stream of nitrogen gas for 30 minutes, and then transferred to a large round bottom flask. Most of the volatiles were removed under reduced pressure with caution so as to avoid foaming and bumping. If water was still present the residue was frozen and lyophilized to dryness. The lyophilized crude mixture was taken up in approximately 50 ml of CH₃CN/10 mM aqueous triethylammonium acetate (60/40). After 0.45 micron PTFE filtration, 4-5 ml sample portions were applied to a C-18 Dynamax column (40×250 mm). Elution was performed with a gradient of acetonitrile and 10 mM aqueous triethylammonium acetate (30% to 50% CH₃CN over 20 minutes at 50 ml/min flow). Fractions from the preparative HPLC runs containing pure (6) were pooled, evaporated to remove CH₃CN and lyophilized to give 360 mg of pure (6) (the RpRp diastereoisomer) as the bis-triethylammonium salt.

To 270 mg (0.24 mmol) of (6) was added 5.0 ml of neat trimethylamine trihydrofluoride. The mixture was stirred at room temperature for approximately 40 h. After confirming completion of reaction by analytical HPLC, the sample was neutralized by dropwise addition into 45 ml of chilled, stirred 1M triethylammonium bicarbonate. The neutralized solution was desalted on a Waters C-18 Sep-Pak and the product eluted with CH₃CN/10 mM aqueous triethylammonium acetate (5:1).The CH₃CN was evaporated under reduced pressure and the remaining aqueous solution was frozen and lyophilized. Multiple rounds of lyophilization from water gave 122 mg (57%) of (T1-2) as the bis-triethylammonium salt. ¹H NMR (500 MHz, 45° C., (CD₃)₂SO-15 μL D₂O) δ 8.58 (s, 1H), 8.41 (s, 1H), 8.18 (s, 1H), 8.15 (s, 1H), 6.12 (d, J=8.0, 1H), 5.92 (d, J=7.0, 1H), 5.30 (td, J=8.5, 4.0, 1H), 5.24-5.21 (m,1H), 5.03 (dd, J=7.5, 4.5, 1H), 4.39 (d, J=4, 1H), 4.23 (dd, J=10.5, 4.0, 1H), 4.18 (s,1H), 4.14-4.08 (m, 2H), 3.85-3.83 (m, 1H), 3.73 (d, J=12.0, 1H), 3.06 (q, J=7.5,12H), 1.15 (t, J=7.5, 1H); ³¹P NMR (200 MHz, 45° C., (CD₃)ISO-15 pL D₂O) 6 58.81, 52.54; HRMS (FT-ICR) I/z calcd for C20H24O10N10P2S2 (M−H) 689.0521, found 689.0514.

An example of a STING agonist assay is as follows. HEK-293T cells were reverse transfected with a mixture of human STING (accession BC047779 with Arg mutation introduced at position 232 to make the clone into human STING wild type) and a 5xISRE-mIFNb-GL4 plasmid (five interferon stimulated response elements and a minimal mouse interferon beta promoter driving expression of the firefly luciferase GL4). Cells were transfected using FuGENE transfection reagent (3:1 FuGENE:DNA ratio) by adding the FuGENE:DNA mix to HEK-293T cells in suspension and plating into 384 well plates. Cells were incubated overnight and treated with compounds. After 9-14 hours, plates were read by adding BrightGlo reagent (Promega) and reading on an Envision plate reader. The fold change over background was calculated and normalized to the fold-change induced by 2′3′-cGAMP at 50 μM. Plates were run in triplicate. EC50 values were calculated as described for the IP-10 secretion assay.

### IL-15

As used herein, the terms “IL-15” and “interleukin-15” refer to a wild-type IL-15 or an IL-15 derivative. In specific embodiments, the IL-15 is isolated and recombinantly produced. As used herein, the terms “wild-type IL-15” and “wild type interleukin-15” in the context of proteins or polypeptides refer to any mammalian interleukin-15 amino acid sequences, including immature or precursor and mature forms. Non-limiting examples of GeneBank Accession Nos. for the amino acid sequence of various species of wild type mammalian interleukin-15 include NP_000576 (human), CAA62616 (human), AAI00964 (human), AAH18149 (human), NP_001009207 (Fells catus), AAB94536 (Rattus norvegicus), AAB41697 (Rattus norvegicus), NP_032383 (Mus musculus), AAH23698 (mus musculus), AAR19080 (canine) and AAB60398 (Macaca mulatta). The amino acid sequence of the immature/precursor form of human IL-15, which comprises the long signal peptide (underlined) and the mature human IL-15 (italicized), as provided in SEQ ID NO: 1. In some embodiments, the IL-15 is the immature or precursor form of a mammalian IL-15. In other embodiments, the IL-15 is the mature form of a mammalian IL-15. In a specific embodiment, the IL-15 is the precursor form of human IL-15. In another embodiment, the IL-15 is the mature form of human IL-15. In one embodiment, the IL-15 protein/polypeptide is isolated or purified.

As used herein, the terms “IL-15” and “interleukin-15” in the context of nucleic acids refer to any nucleic acid sequences encoding mammalian interleukin-15, including the immature or precursor and mature forms. Non-limiting examples of GeneBank Accession Nos. for the nucleotide sequence of various species of wild type mammalian IL-15 include NM_000585 (human), NM_008357 (Mus musculus), and RNU69272 (Rattus norvegicus). The nucleotide sequence encoding the immature/precursor form of human IL-15, which comprises the nucleotide sequence encoding the long signal peptide (underlined) and the nucleotide sequence encoding the mature human IL-15 (italicized), as provided in SEQ ID NO: 2. In a specific embodiment, the nucleic acid is an isolated or purified nucleic acid. In some embodiments, nucleic acids encode the immature or precursor form of a mammalian IL-15. In other embodiments, nucleic acids encode the mature form of a mammalian IL-15. In a specific embodiment, nucleic acids encoding IL-15 encode the precursor form of human IL-15. In another embodiment, nucleic acids encoding IL-15 encode the mature form of human IL-15.

As used herein, the terms “IL-15 derivative” and “interleukin-15 derivative” in the context of proteins or polypeptides refer to: (a) a polypeptide that is at least 75%, 80%, 85%, 90%, 95%, 98% or 99% identical to a wild-type mammalian IL-15 polypeptide; (b) a polypeptide encoded by a nucleic acid sequence that is at least 75%, 80%, 85%, 90%, 95%, 98% or 99% identical a nucleic acid sequence encoding a wild-type mammalian IL-15 polypeptide; (c) a polypeptide that contains 1, 2, 3, 4, 5, 6, 7, 8, 9, 10, 11, 12, 13, 14, 15, 16, 17, 18, 19, 20 or more amino acid mutations (i.e., additions, deletions and/or substitutions) relative to wild-type mammalian IL-15 polypeptide; (d) a polypeptide encoded by nucleic acids can hybridize under high or moderate stringency hybridization conditions to nucleic acids encoding a wild-type mammalian IL-15 polypeptide; (e) a polypeptide encoded by a nucleic acid sequence that can hybridize under high or moderate stringency hybridization conditions to a nucleic acid sequence encoding a fragment of a wild-type mammalian IL-15 polypeptide of at least 20 contiguous amino acids, at least 30 contiguous amino acids, at least 40 contiguous amino acids, at least 50 contiguous amino acids, at least 100 contiguous amino acids, or at least 150 contiguous amino acids; and/or (f) a fragment of a mammalian IL-15 polypeptide. IL-15 derivatives also include a polypeptide that comprises the amino acid sequence of a mature form of a mammalian IL-15 polypeptide and a heterologous signal peptide amino acid sequence. In a specific embodiment, an IL-15 derivative is a derivative of a wild-type human IL-15 polypeptide. In another embodiment, an IL-15 derivative is a derivative of an immature or precursor form of human IL-15 polypeptide. In another embodiment, an IL-15 derivative is a derivative of a mature form of human IL-15 polypeptide. In another embodiment, an IL-15 derivative is the IL-15N72D described in, e.g., Zhu et al., (2009), J. Immunol. 183: 3598 or U.S. Pat. No. 8,163,879. In another embodiment, an IL-15 derivative is one of the IL-15 variants described in U.S. Pat. No. 8,163,879. In one embodiment, an IL-15 derivative is isolated or purified.

In a preferred embodiment, IL-15 derivatives retain at least 75%, 80%, 85%, 90%, 95%, 98% or 99% of the function of wild-type mammalian IL-15 polypeptide to bind IL-15Ra polypeptide, as measured by assays well known in the art, e.g., ELISA, BIAcore®, co-immunoprecipitation. In another preferred embodiment, IL-15 derivatives retain at least 75%, 80%, 85%, 90%, 95%, 98% or 99% of the function of wild-type mammalian IL-15 polypeptide to induce IL-15-mediated signal transduction, as measured by assays well-known in the art, e.g., electromobility shift assays, ELISAs and other immunoassays. In a specific embodiment, IL-15 derivatives bind to IL-15Ra and/or IL-15Rβγ as assessed by, e.g., ligand/receptor binding assays well-known in the art. Percent identity can be determined using any method known to one of skill in the art and as described supra.

As used herein, the terms “IL-15 derivative” and “interleukin-15 derivative” in the context of nucleic acids refer to: (a) a nucleic acid sequence that is at least 75%, 80%, 85%, 90%, 95%, 98% or 99% identical to the nucleic acid sequence encoding a wild-type mammalian IL-15 polypeptide; (b) a nucleic acid sequence encoding a polypeptide that is at least 75%, 80%, 85%, 90%, 95%, 98% or 99% identical the amino acid sequence of a wild-type mammalian IL-15 polypeptide; (c) a nucleic acid sequence that contains 1, 2, 3, 4, 5, 6, 7, 8, 9, 10, 11, 12, 13, 14, 15, 16, 17, 18, 19, 20 or more nucleic acid base mutations (i.e., additions, deletions and/or substitutions) relative to the nucleic acid sequence encoding a mammalian IL-15 polypeptide; (d) a nucleic acid sequence that hybridizes under high or moderate stringency hybridization conditions to a nucleic acid sequence encoding a mammalian IL-15 polypeptide; (e) a nucleic acid sequence that hybridizes under high or moderate stringency hybridization conditions to a fragment of a nucleic acid sequence encoding a mammalian IL-15 polypeptide; and/or (f) a nucleic acid sequence encoding a fragment of a nucleic acid sequence encoding a mammalian IL-15 polypeptide. In a specific embodiment, an IL-15 derivative in the context of nucleic acids is a derivative of a nucleic acid sequence encoding a human IL-15 polypeptide. In another embodiment, an IL-15 derivative in the context of nucleic acids is a derivative of a nucleic acid sequence encoding an immature or precursor form of a human IL-15 polypeptide. In another embodiment, an IL-15 derivative in the context of nucleic acids is a derivative of a nucleic acid sequence encoding a mature form of a human IL-15 polypeptide. In another embodiment, an IL-15 derivative in the context of nucleic acids is the nucleic acid sequence encoding the IL-15N72D described in, e.g., Zhu et al., (2009; supra), or U.S. Pat. No. 8,163,879. In another embodiment, an IL-15 derivative in the context of nucleic acids is the nucleic acid sequence encoding one of the IL-15 variants described in U.S. Pat. No. 8,163,879.

IL-15 derivative nucleic acid sequences include codon-optimized nucleic acid sequences that encode mammalian IL-15 polypeptide, including mature and immature forms of IL-15 polypeptide. In other embodiments, IL-15 derivative nucleic acids include nucleic acids that encode mammalian IL-15 RNA transcripts containing mutations that eliminate potential splice sites and instability elements (e.g., A/T or A/U rich elements) without affecting the amino acid sequence to increase the stability of the mammalian IL-15 RNA transcripts. In one embodiment, the IL-15 derivative nucleic acid sequences include the codon-optimized nucleic acid sequences described in PCT Publication WO2007/084342. In certain embodiments, the IL-15 derivative nucleic acid sequence is the codon-optimized sequence in SEQ ID NO: 4 (the amino acid sequence encoded by such a nucleic acid sequence is provided in SEQ ID NO: 5).

In one embodiment, IL-15 derivative nucleic acid sequences encode proteins or polypeptides that retain at least 75%, 80%, 85%, 90%, 95%, 98% or 99% of the function of a wild-type mammalian IL-15 polypeptide to bind IL-15Ra, as measured by assays well known in the art, e.g., ELISA, BIAcore®, co-immunoprecipitation. In another preferred embodiment, IL-15 derivative nucleic acid sequences encode proteins or polypeptides that retain at least 75%, 80%, 85%, 90%, 95%, 98% or 99% of the function of a wild-type mammalian IL-15 polypeptide to induce IL-15-mediated signal transduction, as measured by assays well-known in the art, e.g., electromobility shift assays, ELISAs and other immunoassays. In a specific embodiment, IL-15 derivative nucleic acid sequences encode proteins or polypeptides that bind to IL-15Ra and/or IL-15Rβγ as assessed by, e.g., ligand/receptor assays well-known in the art.

### IL-15Ra

As used herein, the terms “IL-15Ra” and “interleukin-15 receptor alpha” refer to a wild-type IL-15Ra, an IL-15Ra derivative, or a wild-type IL-15Ra and an IL-15Ra derivative. In specific embodiments, the IL-15Ra is isolated and recombinantly produced. As used herein, the terms “wild-type IL-15Ra” and “wild-type interleukin-15 receptor alpha” in the context of proteins or polypeptides refer to mammalian interleukin-15 receptor alpha (“IL-15Ra”) amino acid sequence, including immature or precursor and mature forms and isoforms. Non-limiting examples of GeneBank Accession Nos. for the amino acid sequence of various wild type mammalian IL-15Ra include NP_002180 (human), ABK41438 (Macaca mulatta), NP_032384 (Mus musculus), Q60819 (Mus musculus), CAI41082 (human). The amino acid sequence of the immature form of the full length human IL-15Ra, which comprises the signal peptide (underlined) and the mature human wild type IL-15Ra (italicized), as provided in SEQ ID NO: 6. The amino acid sequence of the immature form of the soluble human IL-15Ra, which comprises the signal peptide (underlined) and the mature human soluble IL-15Ra (italicized), as provided in SEQ ID NO: 7. In some embodiments, IL-15Ra is the immature form of a mammalian IL-15Ra polypeptide. In other embodiments, the IL-15Ra is the mature form of a mammalian IL-15Ra polypeptide. In certain embodiments, the IL-15Ra is the soluble form of mammalian IL-15Ra polypeptide. In other embodiments, the IL-15Ra is the full-length form of a mammalian IL-15Ra polypeptide. In a specific embodiment, the IL-15Ra is the immature form of a human IL-15Ra polypeptide. In another embodiment, the IL-15Ra is the mature form of a human IL-15Ra polypeptide. In certain embodiments, the IL-15Ra is the soluble form of human IL-15Ra polypeptide. In other embodiments, the IL-15Ra is the full-length form of a human IL-15Ra polypeptide. In one embodiment, the IL-15Ra protein or polypeptide is isolated or purified.

As used herein, the terms “IL-15Ra” and “interleukin-15 receptor alpha” in the context of nucleic acids refer to any nucleic acid sequences encoding mammalian interleukin-15 receptor alpha, including the immature or precursor and mature forms. Non-limiting examples of GeneBank Accession Nos. for the nucleotide sequence of various species of wild type mammalian IL-15Ra include NM_002189 (human), EF033114 (Macaca mulatta), and NM_008358 (Mus musculus). The nucleotide sequence encoding the immature form of human IL-15Ra, which comprises the nucleotide sequence encoding the signal peptide (underlined) and the nucleotide sequence encoding the mature human IL-15Ra (italicized), as provided in SEQ ID NO: 8. The nucleotide sequence encoding the immature form of soluble human IL-15Ra protein or polypeptide, which comprises the nucleotide sequence encoding the signal peptide (underlined) and the nucleotide sequence encoding the mature human soluble IL-15Ra (italicized), as provided in SEQ ID NO: 9. In a specific embodiment, the nucleic acid is an isolated or purified nucleic acid. In some embodiments, nucleic acids encode the immature form of a mammalian IL-15Ra polypeptide. In other embodiments, nucleic acids encode the mature form of a mammalian IL-15Ra polypeptide. In certain embodiments, nucleic acids encode the soluble form of a mammalian IL-15Ra polypeptide. In other embodiments, nucleic acids encode the full-length form of a mammalian IL-15Ra polypeptide. In a specific embodiment, nucleic acids encode the precursor form of a human IL-15 polypeptide. In another embodiment, nucleic acids encode the mature of human IL-15 polypeptide. In certain embodiments, nucleic acids encode the soluble form of a human IL-15Ra polypeptide. In other embodiments, nucleic acids encode the full-length form of a human IL-15Ra polypeptide.

As used herein, the terms “IL-15Ra derivative” and “interleukin-15 receptor alpha derivative” in the context of a protein or polypeptide refer to: (a) a polypeptide that is at least 75%, 80%, 85%, 90%, 95%, 98% or 99% identical to a wild-type mammalian IL-15 polypeptide; (b) a polypeptide encoded by a nucleic acid sequence that is at least 75%, 80%, 85%, 90%, 95%, 98% or 99% identical to a nucleic acid sequence encoding a wild-type mammalian IL-15Ra polypeptide; (c) a polypeptide that contains 1, 2, 3, 4, 5, 6, 7, 8, 9, 10, 11, 12, 13, 14, 15, 16, 17, 18, 19, 20 or more amino acid mutations (i.e., additions, deletions and/or substitutions) relative to the wild-type mammalian IL-15Ra polypeptide; (d) a polypeptide encoded by a nucleic acid sequence that can hybridize under high or moderate stringency hybridization conditions to a nucleic acid sequence encoding a wild-type mammalian IL-15Ra polypeptide; (e) a polypeptide encoded by a nucleic acid sequence that can hybridize under high or moderate stringency hybridization conditions to nucleic acid sequences encoding a fragment of a wild-type mammalian IL-15 polypeptide of at least 20 contiguous amino acids, at least 30 contiguous amino acids, at least 40 contiguous amino acids, at least 50 contiguous amino acids, at least 100 contiguous amino acids, or at least 150 contiguous amino acids; (f) a fragment of a wild type mammalian IL-15Ra polypeptide; and/or (g) a specific IL-15Ra derivative described herein. IL-15Ra derivatives also include a polypeptide that comprises the amino acid sequence of a mature form of mammalian IL-15Ra polypeptide and a heterologous signal peptide amino acid sequence. In a specific embodiment, an IL-15Ra derivative is a derivative of a wild type human IL-15Ra polypeptide. In another embodiment, an IL-15Ra derivative is a derivative of an immature form of human IL-15 polypeptide. In another embodiment, an IL-15Ra derivative is a derivative of a mature form of human IL-15 polypeptide. In one embodiment, an IL-15Ra derivative is a soluble form of a mammalian IL-15Ra polypeptide. In certain embodiments, an IL-15Ra derivative includes soluble forms of mammalian IL-15Ra, wherein those soluble forms are not naturally occurring. Other examples of IL-15Ra derivatives include the truncated, soluble forms of human IL-15Ra described herein. In a specific embodiment, an IL-15Ra derivative is purified or isolated.

In a preferred embodiment, IL-15Ra derivatives retain at least 75%, 80%, 85%, 90%, 95%, 98% or 99% of the function of a wild-type mammalian IL-15Ra polypeptide to bind to an IL-15 polypeptide, as measured by assays well known in the art, e.g., ELISA, BIAcore®, co-immunoprecipitation. In another preferred embodiment, IL-15Ra derivatives retain at least 75%, 80%, 85%, 90%, 95%, 98% or 99% of the function of a wild-type mammalian IL-15Ra polypeptide to induce IL-15-mediated signal transduction, as measured by assays well-known in the art, e.g., electromobility shift assays, ELISAs and other immunoassays. In a specific embodiment, IL-15Ra derivatives bind to an IL-15 as assessed by methods well-known in the art, such as, e.g., ELISAs.

As used herein, the terms “IL-15Ra derivative” and “interleukin-15 receptor alpha derivative” in the context of nucleic acids refer to: (a) a nucleic acid sequence that is at least 75%, 80%, 85%, 90%, 95%, 98% or 99% identical to the nucleic acid sequence encoding a mammalian IL-15Ra polypeptide; (b) a nucleic acid sequence encoding a polypeptide that is at least 75%, 80%, 85%, 90%, 95%, 98% or 99% identical the amino acid sequence of a wild type mammalian IL-15Ra polypeptide; (c) a nucleic acid sequence that contains 1, 2, 3, 4, 5, 6, 7, 8, 9, 10, 11, 12, 13, 14, 15, 16, 17, 18, 19, 20 or more nucleic acid mutations (i.e., additions, deletions and/or substitutions) relative to the nucleic acid sequence encoding a mammalian IL-15Ra polypeptide; (d) a nucleic acid sequence that hybridizes under high or moderate stringency hybridization conditions to a nucleic acid sequence encoding a mammalian IL-15Ra polypeptide; (e) a nucleic acid sequence that hybridizes under high or moderate stringency hybridization conditions to a fragment of a nucleic acid sequence encoding a mammalian IL-15Ra polypeptide; (f) a nucleic acid sequence encoding a fragment of a nucleic acid sequence encoding a mammalian IL-15Ra polypeptide; and/or (g) a nucleic acid sequence encoding a specific IL-15Ra derivative described herein. In a specific embodiment, an IL-15Ra derivative in the context of nucleic acids is a derivative of a nucleic acid sequence encoding a human IL-15Ra polypeptide. In another embodiment, an IL-15Ra derivative in the context of nucleic acids is a derivative of a nucleic acid sequence encoding an immature form of a human IL-15Ra polypeptide. In another embodiment, an IL-15Ra derivative in the context of nucleic acids is a derivative of a nucleic acid sequence encoding a mature form of a human IL-15Ra polypeptide. In one embodiment, an IL-15Ra derivative in the context of nucleic acids refers to a nucleic acid sequence encoding a derivative of mammalian IL-15Ra polypeptide that is soluble. In certain embodiments, an IL-15Ra derivative in context of nucleic acids refers to a nucleic acid sequence encoding a soluble form of mammalian IL-15Ra, wherein the soluble form is not naturally occurring. In some embodiments, an IL-15Ra derivative in the context of nucleic acids refers to a nucleic acid sequence encoding a derivative of human IL-15Ra, wherein the derivative of the human IL-15Ra is a soluble form of IL-15Ra that is not naturally occurring. In specific embodiments, an IL-15Ra derivative nucleic acid sequence is isolated or purified.

IL-15Ra derivative nucleic acid sequences include codon-optimized nucleic acid sequences that encode a IL-15Ra polypeptide, including mature and immature forms of IL-15Ra polypeptide. In other embodiments, IL-15Ra derivative nucleic acids include nucleic acids that encode IL-15Ra RNA transcripts containing mutations that eliminate potential splice sites and instability elements (e.g., A/T or A/U rich elements) without affecting the amino acid sequence to increase the stability of the IL-15Ra RNA transcripts. In certain embodiments, the IL-15Ra derivative nucleic acid sequence is the codon-optimized sequence in SEQ ID NO: 11 or SEQ ID NO: 13 (the amino acid sequences encoded by such a nucleic acid sequences are provided in SEQ ID NO: 12 and SEQ ID NO: 14, respectively).

In specific embodiments, IL-15Ra derivative nucleic acid sequences encode proteins or polypeptides that retain at least 50%, 55%, 60%, 65%, 70%, 75%, 80%, 85%, 90%, 95%, 98% or 99% of the function of a wild type mammalian IL-15Ra polypeptide to bind IL-15, as measured by assays well known in the art, e.g., ELISA, BIAcore®, co-immunoprecipitation. In another preferred embodiment, IL-15Ra derivative nucleic acid sequences encode proteins or polypeptides that retain at least 50%, 55%, 60%, 65%, 70%, 75%, 80%, 85%, 90%, 95%, 98% or 99% of the function of a wild type mammalian IL-15Ra to induce IL-15-mediated signal transduction, as measured by assays well-known in the art, e.g., electromobility shift assays, ELISAs and other immunoassays. In a specific embodiment, IL-15Ra derivative nucleic acid sequences encode proteins or polypeptides that bind to IL-15 as assessed by methods well-known in the art, such as, e.g., ELISAs.

Described herein is a soluble form of human IL-15Ra. Also described herein are specific IL-15Ra derivatives that are truncated, soluble forms of human IL-15Ra. These specific IL-15Ra derivatives and the soluble form of human IL-15Ra are based, in part, on the identification of the proteolytic cleavage site of human IL-15Ra. Further described herein are soluble forms of IL-15Ra that are characterized based upon glycosylation of the IL-15Ra.

The proteolytic cleavage of human IL-15Ra takes place between the residues (i.e., Gly170 and His171) which are in shown in bold and underlined in the provided amino acid sequence of the immature form of the wild-type full length human IL-15Ra:

(SEQ ID NO: 6)
MAPRRARGCRTLGLPALLLLLLLRPPATRGITCPPP
MSVEHADIWVKSYSLYSRERYICNSGFKRKAGTSS
LTECVLNKATNVAHWTTPSLKCIRDPALVHQRPAP
PSTVTTAGVTPQPESLSPSGKEPAASSPSSNNTAA
TTAAIVPGSQLMPSKSPSTGTTEISSHESSHGTPS
QTTAKNWELTASASHQPPGVYPQGHSDTTVAISTS
TVLLCGLSAVSLLACYLKSRQTPPLASVEMEAMEA
LPVTWGTSSRDEDLENCSHHL.

Accordingly, in one aspect, provided herein is a soluble form of human IL-15Ra (e.g., a purified soluble form of human IL-15Ra), wherein the amino acid sequence of the soluble form of human IL-15Ra terminates at the site of the proteolytic cleavage of the wild-type membrane-bound human IL-15Ra. In particular, provided herein is a soluble form of human IL-15Ra (e.g., a purified soluble form of human IL-15Ra), wherein the amino acid sequence of the soluble form of human IL-15Ra terminates with PQG (SEQ ID NO: 20), wherein G is Gly170. In a particular embodiment, provided herein is a soluble form of human IL-15Ra (e.g., a purified soluble form of human IL-15Ra) which has the amino acid sequence shown in SEQ ID NO: 7. In some embodiments, provided herein is an IL-15Ra derivative (e.g., a purified and/or soluble form of IL-15Ra derivative), which is a polypeptide that: (i) is at least 75%, 80%, 85%, 90%, 95%, 98% or 99% identical to SEQ ID NO: 7; and (ii) terminates with the amino acid sequence PQG (SEQ ID NO: 20). In other particular embodiments, provided herein is a soluble form of human IL-15Ra (e.g., a purified soluble form of human IL-15Ra) which has the amino acid sequence of SEQ ID NO: 10). In some embodiments, provided herein is an IL-15Ra derivative (e.g., a purified and/or soluble form of an IL-15Ra derivative), which is a polypeptide that is at least 75%, 80%, 85%, 90%, 95%, 98% or 99% identical to SEQ ID NO: 10, and, optionally, wherein the amino acid sequence of the soluble form of the IL-15Ra derivative terminates with PQG (SEQ ID NO: 20).

In some embodiments, provided herein is an IL-15Ra derivative of human IL-15Ra, wherein the IL-15Ra derivative is soluble and: (a) the last amino acids at the C-terminal end of the IL-15Ra derivative consist of amino acid residues PQGHSDTT (SEQ ID NO: 15); (b) the last amino acids at the C-terminal end of the IL-15Ra derivative consist of amino acid residues PQGHSDT (SEQ ID NO: 16); (c) the last amino acids at the C-terminal end of the IL-15Ra derivative consist of amino acid residues PQGHSD (SEQ ID NO: 17); (d) the last amino acids at the C-terminal end of the IL-15Ra derivative consist of amino acid residues PQGHS (SEQ ID NO: 18); or (e) the last amino acids at the C-terminal end of the IL-15Ra derivative consist of amino acid residues PQGH (SEQ ID NO: 19). In certain embodiments, the amino acid sequences of these IL-15Ra derivatives are at least 75%, at least 85%, at least 90%, at least 95%, at least 96%, at least 97%, at least 98%, or at least 99% identical to the amino acid sequence of SEQ ID NO: 21. In some embodiments, these IL-15Ra derivatives are purified.

In another aspect, provided herein are glycosylated forms of IL-15Ra (e.g., purified glycosylated forms of IL-15Ra), wherein the glycosylation of the IL-15Ra accounts for at least 20%, at least 25%, at least 30%, at least 35%, at least 40%, at least 45%, at least 50%, or 20% to 25%, 20% to 30%, 25% to 30%, 25% to 35%, 30% to 35%, 30% to 40%, 35% to 40%, 35% to 45%, 40% to 50%, 45% to 50%, 20% to 40%, or 25% to 50% of the mass (molecular weight) of the IL-15Ra as assessed by techniques known to one of skill in the art. The percentage of the mass (molecular weight) of IL-15Ra (e.g., purified IL-15Ra) that glycosylation of IL-15Ra accounts for can be determined using, for example and without limitation, gel electrophoresis and quantitative densitometry of the gels, and comparison of the average mass (molecular weight) of a glycosylated form of IL-15Ra (e.g., a purified glycosylated form of IL-15Ra) to the non-glycosylated form of IL-15Ra (e.g., a purified non-glycosylated form of IL-15Ra). In one embodiment, the average mass (molecular weight) of IL-15Ra (e.g., purified IL-15Ra) can be determined using MALDI-TOF MS spectrum on Voyager De-Pro® equipped with CovalX HM-1 high mass detector using sinapic acid as matrix, and the mass of a glycosylated form of IL-15Ra (e.g., purified glycosylated form of IL-15Ra) can be compared to the mass of the non-glycosylated form of IL-15Ra (e.g., purified non-glycosylated form of IL-15Ra) to determine the percentage of the mass that glycosylation accounts for.

In another aspect, provided herein are glycosylated forms of IL-15Ra, wherein the IL-15Ra is glycosylated (N- or O-glycosylated) at certain amino acid residues. In certain embodiments, provided herein is a human IL-15Ra which is glycosylated at one, two, three, four, five, six, seven, or all, of the following glycosylation sites:
(i) O-glycosylation on threonine at position 5 of the amino acid sequence NWELTASASHQPPGVYPQG (SEQ ID NO: 22) in the IL-15Ra;
(ii) O-glycosylation on serine at position 7 of the amino acid sequence NWELTASASHQPPGVYPQG (SEQ ID NO: 22) in the IL-15Ra;
(iii) N-glycosylation on serine at position 8 of the amino acid sequence ITCPPPMSVEHADIWVK (SEQ ID NO: 23) in the IL-15Ra, or serine at position 8 of the amino acid sequence ITCPPPMSVEHADIWVKSYSLYSRERYICNS (SEQ ID NO: 24) in the IL-15Ra;
(iv) N-glycosylation on Ser 18 of amino acid sequence ITCPPPMSVEHADIWVKSYSLYSRERYICNS (SEQ ID NO: 24) in the IL-15Ra;
(v) N-glycosylation on serine at position 20 of the amino acid sequence ITCPPPMSVEHADIWVKSYSLYSRERYICNS (SEQ ID NO: 24) in the IL-15Ra;
(vi) N-glycosylation on serine at position 23 of the amino acid sequence ITCPPPMSVEHADIWVKSYSLYSRERYICNS (SEQ ID NO: 24) in the IL-15Ra;
and/or (vii) N-glycosylated on serine at position 31 of the amino acid sequence ITCPPPMSVEHADIWVKSYSLYSRERYICNS (SEQ ID NO: 24) in the IL-15Ra.

In specific embodiments, the glycosylated IL-15Ra is the wild-type human IL-15Ra. In other specific embodiments, the glycosylated IL-15Ra is an IL-15Ra derivative of human IL-15Ra. In some embodiments, the glycosylated IL-15Ra is a wild-type soluble human IL-15Ra, such as SEQ ID NO: 7 or SEQ ID NO: 10. In other embodiments, the glycosylated IL-15Ra is an IL-15Ra derivative that is a soluble form of human IL-15Ra. In certain embodiments, the glycosylated IL-15Ra is purified or isolated.

### IL-15/IL-15Ra Complex

As used herein, the term “IL-15/IL-15Ra complex” refers to a complex comprising IL-15 and IL-15Ra covalently or noncovalently bound to each other. In a preferred embodiment, the IL-15Ra has a relatively high affinity for IL-15, e.g., KD of 10 to 50 pM as measured by a technique known in the art, e.g., KinEx A assay, plasma surface resonance (e.g., BIAcore® assay). In another preferred embodiment, the IL-15/IL-15Ra complex induces IL-15-mediated signal transduction, as measured by assays well-known in the art, e.g., electromobility shift assays, ELISAs and other immunoassays. In some embodiments, the IL-15/IL-15Ra complex retains the ability to specifically bind to the 13y chain In a specific embodiment, the IL-15/IL-15Ra complex is isolated from a cell.

Provided herein are complexes that bind to the βγ subunits of the IL-15 receptor, induce IL-15 signal transduction (e.g., Jak/Stat signal transduction) and enhance IL-15-mediated immune function, wherein the complexes comprise IL-15 covalently or noncovalently bound to interleukin-15 receptor alpha (“IL-15Ra”) (a “IL-15/IL-15Ra complex”). The IL-15/IL-15Ra complex is able to bind to the βγ receptor complex.

The IL-15/IL-15Ra complexes can be comprised of a wild type IL-15 or an IL-15 derivative and a wild type IL-15Ra or an IL-15Ra derivative. In certain embodiments, an IL-15/IL-15Ra complex comprises a wild type IL-15 or an IL-15 derivative and an IL-15Ra described above. In a specific embodiment, an IL-15/IL-15Ra complex comprises a wild type IL-15 or an IL-15 derivative and IL-15Ra with the amino acid sequence of SEQ ID NO: 10. In another embodiment, an IL-15/IL-15Ra complex comprises wild type IL-15 or an IL-15 derivative and a glycosylated form of IL-15Ra described supra.

In a specific embodiment, an IL-15/IL-15Ra complex comprises a wild type IL-15 or an IL-15Ra derivative and a soluble IL-15Ra. In another specific embodiment, an IL-15/IL-15Ra complex is composed of an IL-15 derivative and an IL-15Ra derivative. In another embodiment, an IL-15/IL-15Ra complex is composed of wild type IL-15 and an IL-15Ra derivative. In one embodiment, the IL-15Ra derivative is a soluble form of IL-15Ra. Specific examples of soluble forms of IL-15Ra are described above. In a specific embodiment, the soluble form of IL-15Ra lacks the transmembrane domain of wild type IL-15Ra, and optionally, the intracellular domain of wild type IL-15Ra. In another embodiment, the IL-15Ra derivative is the extracellular domain of IL-15Ra or a fragment thereof. In certain embodiments, the IL-15Ra derivative is a fragment of the extracellular domain comprising the sushi domain or exon 2 of IL-15Ra. In some embodiments, the IL-15Ra derivative comprises a fragment of the extracellular domain comprising the sushi domain or exon 2 of IL-15Ra and at least one amino acid that is encoded by exon 3. In certain embodiments, the IL-15Ra derivative comprises a fragment of the extracellular domain comprising the sushi domain or exon 2 of IL-15Ra and an IL-15Ra hinge region or a fragment thereof. In certain embodiments, the IL-15Ra comprises the amino acid sequence of SEQ ID NO: 10.

In another embodiment, the IL-15Ra derivative comprises a mutation in the extracellular domain cleavage site that inhibits cleavage by an endogenous protease that cleaves wild type IL-15Ra. In a specific embodiment, the extracellular domain cleavage site of IL-15Ra is replaced with a cleavage site that is recognized and cleaved by a heterologous known protease. Non-limiting examples of such heterologous protease cleavage sites include Arg-X-X-Arg (SEQ ID NO: 25), which is recognized and cleaved by furin protease; and A-B-Pro-Arg-X-Y (SEQ ID NO: 26) (A and B are hydrophobic amino acids and X and Y are non-acidic amino acids) and Gly-Arg-Gly, which are recognized and cleaved by thrombin protease.

In another embodiment, the IL-15 is encoded by a nucleic acid sequence optimized to enhance expression of IL-15, e.g., using methods as described in PCT Publications WO 2007/084342 and WO 2010/020047; and U.S. Pat. Nos. 5,965,726; 6,174,666; 6,291,664; 6,414,132; and 6,794,498.

In certain embodiments, provided herein is an IL-15/IL-15Ra complex comprising human IL-15Ra which is glycosylated at one, two, three, four, five, six, seven, or all, of the glycosylation sites as described supra and with reference to SEQ ID NOs: 22, 23 and 24. In specific embodiments, the glycosylated IL-15Ra is a wild type human IL-15Ra. In other specific embodiments, the glycosylated IL-15Ra is an IL-15Ra derivative of human IL-15Ra. In some embodiments, the glycosylated IL-15Ra is a soluble human IL-15Ra, such as SEQ ID NO: 7 or SEQ ID NO: 10. As used herein, “hetIL15” is human IL-15 comprising residues 49 to 162 of the amino acid sequence of SEQ ID NO: 1 and the human soluble IL-15Ra comprising the amino acid sequence of SEQ ID NO: 10. In other embodiments, the glycosylated IL-15Ra is an IL-15Ra derivative that is a soluble form of human IL-15Ra. In certain embodiments, the IL-15/IL-15Ra complex is purified or isolated.

In addition to IL-15 and IL-15Ra, the IL-15/IL-15Ra complexes can comprise a heterologous molecule. In some embodiments, the heterologous molecule increases protein stability. Non-limiting examples of such molecules include polyethylene glycol (PEG), Fc domain of an IgG immunoglobulin or a fragment thereof, or albumin that increase the half-life of IL-15 or IL-15Ra in vivo. In certain embodiments, IL-15Ra is conjugated/fused to the Fc domain of an immunoglobulin (e.g., an IgG1) or a fragment thereof. In a specific embodiment, the IL-15RaFc fusion protein comprises the amino acid sequence of SEQ ID NO: 27 or SEQ ID NO: 28. In another embodiment, the IL-15RaFc fusion protein is the IL-15Ra/Fc fusion protein described in Han et al., (2011), Cytokine 56: 804-810, U.S. Pat. No. 8,507,222 or U.S. Pat. No. 8,124,084. In those IL-15/IL-15Ra complexes comprising a heterologous molecule, the heterologous molecule can be conjugated to IL-15 and/or IL-15Ra. In one embodiment, the heterologous molecule is conjugated to IL-15Ra. In another embodiment, the heterologous molecule is conjugated to IL-15.

The components of an IL-15/IL-15Ra complex can be directly fused, using either non-covalent bonds or covalent bonds (e.g., by combining amino acid sequences via peptide bonds), and/or can be combined using one or more linkers. Linkers suitable for preparing the IL-15/IL-15Ra complexes comprise peptides, alkyl groups, chemically substituted alkyl groups, polymers, or any other covalently-bonded or non-covalently bonded chemical substance capable of binding together two or more components. Polymer linkers comprise any polymers known in the art, including polyethylene glycol (PEG). In some embodiments, the linker is a peptide that is 1, 2, 3, 4, 5, 6, 7, 8, 9, 10, 11, 12, 13, 14, 15, 16, 17, 18, 19, 20 or more amino acids long. In a specific embodiment, the linker is long enough to preserve the ability of IL-15 to bind to the IL-15Ra. In other embodiments, the linker is long enough to preserve the ability of the IL-15/IL-15Ra complex to bind to the βγ receptor complex and to act as an agonist to mediate IL-15 signal transduction.

In particular embodiments, IL-15/IL-15Ra complexes are pre-coupled prior to administration in the methods described herein (e.g., prior to contacting cells with the IL-15/IL-15Ra complexes or prior to administering the IL-15/IL-15Ra complexes to a subject). In other embodiments, the IL-15/IL-15Ra complexes are not pre-coupled prior to use in the methods described herein.

In a specific embodiment, an IL-15/IL-15Ra complex enhances or induces immune function in a subject by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to the immune function in a subject not administered the IL-15/IL-15Ra complex using assays well known in the art, e.g., ELISPOT, ELISA, and cell proliferation assays. In a specific embodiment, the immune function is cytokine release (e.g., interferon-gamma, IL-2, IL-5, IL-10, IL-12, or transforming growth factor (TGF) -beta). In one embodiment, the IL-15 mediated immune function is NK cell proliferation, which can be assayed, e.g., by flow cytometry to detect the number of cells expressing markers of NK cells (e.g., CD56). In another embodiment, the IL-15 mediated immune function is antibody production, which can be assayed, e.g., by ELISA. In some embodiments, the IL-15 mediated immune function is effector function, which can be assayed, e.g., by a cytotoxicity assay or other assays well known in the art.

### Methods of Producing Polypeptides of the Combination

Also provided are expression vectors and host cells for producing the IL-15/IL-15Ra complexes of the combination, as described above. Various expression vectors can be employed to express the polynucleotides encoding the IL-15 and IL-15Ra polypeptides. Both viral-based and nonviral expression vectors can be used to produce the polypeptides in a mammalian host cell. Non-viral vectors and systems include plasmids, episomal vectors, typically with an expression cassette for expressing a protein or RNA, and human artificial chromosomes (see, e.g., Harrington et al., (1997) Nat Genet. 15:345). For example, nonviral vectors useful for expression of polypeptides in mammalian (e.g., human) cells include pThioHis A, B & C, pcDNA3.1/His, pEBVHis A, B & C, (Invitrogen, San Diego, Calif.), MPSV vectors, and numerous other vectors known in the art for expressing other proteins. Useful viral vectors include vectors based on retroviruses, adenoviruses, adenoassociated viruses, herpes viruses, vectors based on SV40, papilloma virus, HBP Epstein Barr virus, vaccinia virus vectors and Semliki Forest virus (SFV). See, Brent et al., supra; Smith, (1995) Annu. Rev. Microbiol. 49:807; and Rosenfeld et al., (1992) Cell 68:143.

The choice of expression vector depends on the intended host cells in which the vector is to be expressed. Typically, the expression vectors contain a promoter and other regulatory sequences (e.g., enhancers) that are operably linked to the polynucleotides. In one embodiment, an inducible promoter is employed to prevent expression of inserted sequences except under inducing conditions. Inducible promoters include, e.g., arabinose, lacZ, metallothionein promoter or a heat shock promoter. Cultures of transformed organisms can be expanded under noninducing conditions without biasing the population for coding sequences whose expression products are better tolerated by the host cells. In addition to promoters, other regulatory elements may also be required or desired for efficient expression. These elements typically include an ATG initiation codon and adjacent ribosome binding site or other sequences. In addition, the efficiency of expression can be enhanced by the inclusion of enhancers appropriate to the cell system in use (see, e.g., Scharf et al., (1994) Results Probl. Cell Differ. 20:125; and Bittner et al., (1987) Meth. Enzymol., 153:516). For example, the SV40 enhancer or CMV enhancer can be used to increase expression in mammalian host cells.

The expression vectors can also provide a secretion signal sequence position to form a fusion protein with polypeptides encoded by the IL-15 or IL-15Ra sequences. More often, the inserted sequences are linked to a signal sequences before inclusion in the vector.

The host cells for harboring and expressing the IL-15 and IL-15Ra proteins can be either prokaryotic or eukaryotic. E. coli is one prokaryotic host useful for cloning and expressing the IL-15 and IL-15Ra polynucleotides. Other microbial hosts suitable for use include bacilli, such as Bacillus subtilis, and other enterobacteriaceae, such as Salmonella, Serratia, and various Pseudomonas species. In these prokaryotic hosts, one can also make expression vectors, which typically contain expression control sequences compatible with the host cell (e.g., an origin of replication). In addition, any number of a variety of well-known promoters will be present, such as the lactose promoter system, a tryptophan (trp) promoter system, a beta-lactamase promoter system, or a promoter system from phage lambda. The promoters typically control expression, optionally with an operator sequence, and have ribosome binding site sequences and the like, for initiating and completing transcription and translation. Other microbes, such as yeast, can also be employed to express IL-15 and IL-15Ra polypeptides. Insect cells in combination with baculovirus vectors can also be used.

In one embodiment, mammalian host cells are used to express and produce the polypeptides of the present disclosure. For example, they can be either a hybridoma cell line expressing endogenous immunoglobulin genes or a mammalian cell line harboring an exogenous expression vector. These include any normal mortal or normal or abnormal immortal animal or human cell. For example, a number of suitable host cell lines capable of secreting intact immunoglobulins have been developed including the CHO cell lines, various Cos cell lines, HeLa cells, myeloma cell lines, transformed B-cells and hybridomas. Examples of mammalian cell lines include, but are not limited to, COS, CHO, HeLa, NIH3T3, HepG2, MCF7, HEK 293, HEK 293T, RD, PC12, hybridomas, pre-B cells, 293, 293H, K562, SkBr3, BT474, A204, M07Sb, TFβ1, Raji, Jurkat, MOLT-4, CTLL-2, MC-IXC, SK-N-MC, SK-N-MC, SK-N-DZ, SH-SY5Y, C127, N0, and BE(2)-C cells. Other mammalian cell lines available as hosts for expression are known in the art and include many immortalized cell lines available from the American Type Culture Collection (ATCC). The use of mammalian tissue cell culture to express polypeptides is discussed generally in, e.g., Winnacker, FROM GENES TO CLONES, VCH Publishers, N.Y., N.Y., 1987.

In another embodiment, the IL-15/IL-15Ra complex is glycosylated by expression in a CHO cell, wherein at least 0.5%, 1%, 2%, 3%, 5% or more of each polypeptide in the complex have an α2,3-linked sialic acid residue. CHO cell expression provides that none of the polypeptides in the IL-15/IL-15Ra complex contain a bisecting GlcNAc.

Expression vectors for mammalian host cells can include expression control sequences, such as an origin of replication, a promoter, and an enhancer (see, e.g., Queen, et al., (1986) Immunol. Rev. 89:49-68,), and necessary processing information sites, such as ribosome binding sites, RNA splice sites, polyadenylation sites, and transcriptional terminator sequences. These expression vectors usually contain promoters derived from mammalian genes or from mammalian viruses. Suitable promoters can be constitutive, cell type-specific, stage-specific, and/or modulatable or regulatable. Useful promoters include, but are not limited to, the metallothionein promoter, the constitutive adenovirus major late promoter, the dexamethasone-inducible MMTV promoter, the SV40 promoter, the MRP poIIII promoter, the constitutive MPSV promoter, the tetracycline-inducible CMV promoter (such as the human immediate-early CMV promoter), the constitutive CMV promoter, and promoter-enhancer combinations known in the art.

Methods for introducing expression vectors containing the polynucleotide sequences of interest vary depending on the type of cellular host. For example, calcium chloride transfection is commonly utilized for prokaryotic cells, whereas calcium phosphate treatment or electroporation can be used for other cellular hosts. (See generally Sambrook et al., supra). Other methods include, e.g., electroporation, calcium phosphate treatment, liposome-mediated transformation, injection and microinjection, ballistic methods, virosomes, immunoliposomes, polycation:nucleic acid conjugates, naked DNA, artificial virions, fusion to the herpes virus structural protein VP22 (Elliot & O'Hare, (1997) Cell 88:223), agent-enhanced uptake of DNA, and ex vivo transduction.

For long-term, high-yield production of recombinant IL-15 and IL-15Ra polypeptides, stable cell lines can be generated. For example, cell lines can be transformed using the nucleic acid constructs described herein which can contain a selectable marker gene on the same or on a separate nucleic acid construct. The selectable marker gene can be introduced into the same cell by co-transfection. Following the introduction of the vector, cells are allowed to grow for 1-2 days in an enriched media before they are switched to selective media to allow growth and recovery of cells that successfully express the introduced nucleic acids. Resistant clones of stably transformed cells can be proliferated using tissue culture techniques well known in the art that are appropriate to the cell type. In a particular embodiment, the cell line has been adapted to grow in serum-free medium. In one embodiment, the cell line has been adapted to grow in serum-free medium in shaker flasks. In one embodiment, the cell line has been adapted to grow in stir or rotating flasks. In certain embodiments, the cell line is cultured in suspension. In particular embodiments, the cell line is not adherent or has been adapted to grow as nonadherent cells. In certain embodiments, the cell line has been adapted to grow in low calcium conditions. In some embodiments, the cell line is cultured or adapted to grow in low serum medium.

In a specific embodiment, a particularly preferred method of high-yield production of a recombinant IL-15 and IL-15Ra polypeptide is through the use of dihydro folate reductase (DHFR) amplification in DHFR-deficient CHO cells, by the use of successively increasing levels of methotrexate as described in U.S. Pat. No. 4,889,803. The polypeptide obtained from such cells can be in a glycosylated form.

In one embodiment, cell lines are engineered to express the stable heterodimer of human IL-15 and soluble human IL-15Ra, which can then be purified, and administered to a human. In one embodiment, the stability of the IL-15/IL-15Ra heterodimer is increased when produced from cell lines recombinantly expressing both IL-15 and IL-15Ra.

In a specific embodiment, the host cell recombinantly expresses IL-15 and the full length IL-15Ra. In another specific embodiment, the host cell recombinantly expresses IL-15 and the soluble form of IL-15Ra. In another specific embodiment, the host cell recombinantly expresses IL-15 and a membrane-bound form of IL-15Ra which is not cleaved from the surface of the cell and remains cell associated. In some embodiments, the host cell recombinantly expressing IL-15 and/or IL-15Ra (full-length or soluble form) also recombinantly expresses another polypeptide (e.g., a cytokine or fragment thereof).

In certain embodiments, such a host cell recombinantly expresses an IL-15 polypeptide in addition to an IL-15Ra polypeptide. The nucleic acids encoding IL-15 and/or IL-15Ra can be used to generate mammalian cells that recombinantly express IL-15 and IL-15Ra in high amounts for the isolation and purification of IL-15 and IL-15Ra, preferably the IL-15 and the IL-15Ra are associated as complexes. In one embodiment, high amounts of IL-15/IL-15Ra complexes refer to amounts of IL-15/IL-15Ra complexes expressed by cells that are at least 1 fold, 2 fold, 3 fold, 4 fold, 5 fold, 6 fold, 7 fold, 8 fold, 9 fold, 10 fold, 20 fold, or more than 20 fold higher than amounts of IL-15/IL-15Ra complexes expressed endogenously by control cells (e.g., cells that have not been genetically engineered to recombinantly express IL-15, IL-15Ra, or both IL-15 and IL-15Ra, or cells comprising an empty vector). In some embodiments, a host cell described herein expresses approximately 0.1 pg to 25 pg, 0.1 pg to 20 pg, 0.1 pg to 15 pg, 0.1 pg to 10 pg, 0.1 pg to 5 pg, 0.1 pg to 2 pg, 2 pg to 10 pg, or 5 to 20 pg of IL-15 as measured by a technique known to one of skill in the art (e.g., an ELISA). In certain embodiments, a host cell described herein expresses approximately 0.1 to 0.25 pg per day, 0.25 to 0.5 pg per day, 0.5 to 1 pg per day, 1 to 2 pg per day, 2 to 5 pg per day, or 5 to 10 pg per day of IL-15 as measured by a technique known to one of skill in the art (e.g., an ELISA). In a specific embodiment, the IL-15Ra is the soluble form of IL-15Ra. In a specific embodiment, the IL-15Ra is the soluble form of IL-15Ra associated with IL-15 in a stable heterodimer, which increases yields and simplifies production and purification of bioactive heterodimer IL-15/soluble IL-15Ra cytokine.

Recombinant IL-15 and IL-15Ra can be purified using methods of recombinant protein production and purification are well known in the art, e.g., see PCT Publication WO 2007/070488. Briefly, the polypeptide can be produced intracellularly, in the periplasmic space, or directly secreted into the medium. Cell lysate or supernatant comprising the polypeptide can be purified using, for example, hydroxylapatite chromatography, gel electrophoresis, dialysis, and affinity chromatography. Other techniques for protein purification such as fractionation on an ion-exchange column, ethanol precipitation, Reverse Phase HPLC, chromatography on silica, chromatography on heparin SEPHAROSE™ (gel filtration substance; Pharmacia Inc., Piscataway, N.J.) chromatography on an anion or cation exchange resin (such as a polyaspartic acid column), chromatofocusing, SDS-PAGE, and ammonium sulfate precipitation are also available.

In some embodiments, IL-15 and IL-15Ra are synthesized or recombinantly expressed by different cells and subsequently isolated and combined to form an IL-15/IL-15Ra complex, in vitro, prior to administration to a subject. In other embodiments, IL-15 and IL-15Ra are synthesized or recombinantly expressed by different cells and subsequently isolated and simultaneously administered to a subject an IL-15/IL-15Ra complex in situ or in vivo. In yet other embodiments, IL-15 and IL-15Ra are synthesized or expressed together by the same cell, and the IL-15/IL-15Ra complex formed is isolated.

### Prophylactic and Therapeutic Uses

The present disclosure provides methods of treating a disease or disorder associated with increased cell proliferation or cancer. In a specific embodiment, the present disclosure provides a method of treating indications including, but not limited to, sarcomas, adenocarcinomas, blastomas, and carcinomas, of the various organ systems, such as those affecting liver, lung, breast, lymphoid, biliarintestinal (e.g., colon), genitourinary tract (e.g., renal, urothelial cells), prostate and pharynx. Adenocarcinomas include malignancies such as most colon cancers, rectal cancer, renal-cell carcinoma, liver cancer, small cell lung cancer, non-small cell carcinoma of the lung, cancer of the small intestine and cancer of the esophagus. In one embodiment, the cancer is a melanoma, e.g., an advanced stage melanoma. Examples of other cancers that can be treated include bone cancer, pancreatic cancer, skin cancer, cancer of the head or neck, cutaneous or intraocular malignant melanoma, uterine cancer, ovarian cancer, rectal cancer, colorectal cancer, cancer of the peritoneum, stomach or gastric cancer, esophageal cancer, salivary gland carcinoma, testicular cancer, uterine cancer, carcinoma of the fallopian tubes, carcinoma of the endometrium, carcinoma of the cervix, carcinoma of the vagina, carcinoma of the vulva, penile carcinoma, glioblastoma, neuroblastoma, cervical cancer, Hodgkin Disease, non-Hodgkin lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, neuroendocrine tumors (including carcinoid tumors, gastrinoma, and islet cell cancer), mesothelioma, schwannoma (including acoustic neuroma), meningioma, epidermoid cancer, squamous cell cancer, T-cell lymphoma, B-cell acute lymphoid leukemia (“BALL”), T-cell acute lymphoid leukemia (“TALL”), acute lymphoid leukemia (ALL); one or more chronic leukemias including but not limited to, e.g., chronic myelogenous leukemia (CML), chronic lymphocytic leukemia (CLL); additional hematologic cancers or hematologic conditions including, but not limited to, e.g., B cell prolymphocytic leukemia, blastic plasmacytoid dendritic cell neoplasm, Burkitt's lymphoma, diffuse large B cell lymphoma, Follicular lymphoma, Hairy cell leukemia, small cell- or a large cell-follicular lymphoma, malignant lymphoproliferative conditions, MALT lymphoma, mantle cell lymphoma, Marginal zone lymphoma, multiple myeloma, myelodysplasia and myelodysplastic syndrome, non-Hodgkin lymphoma, plasmablastic lymphoma and plasmacytoid dendritic cell neoplasm.

Furthermore, the combination of a STING agonist molecule with an IL-15/IL-15Ra complex can be used, inter alia, to treat, prevent, delay or reverse disease progression of patients who have become resistant or refractory to other cancer therapies. By administering the STING agonist molecule, in combination with an IL-15/IL-15Ra complex, anti-tumor immunity can be enhanced, either partially or completely.

In a further embodiment, the combination of a STING agonist molecule with an

IL-15/IL-15Ra complex described herein, can be administered to a patient in need thereof in conjunction with another therapeutic agent as discussed below. For example, the combination of the present disclosure can be co-formulated with, and/or co-administered with, one or more additional therapeutic agents, e.g., one or more anti-cancer agents, cytotoxic or cytostatic agents, hormone treatment, vaccines, and/or other immunotherapies. In other embodiments, the combination can be administered with other therapeutic treatment modalities, including surgery, radiation, cryosurgery, and/or thermotherapy. Such combination therapies can advantageously utilize lower dosages of the administered therapeutic agents, thus avoiding possible toxicities or complications associated with the various monotherapies.

As will be appreciated by the skilled artisan, therapies utilizing the combination of the present disclosure can be administered in conjunction with multiple classes of the agents described above. When the combination of the present disclosure is administered together with another agent or agents, the two (or more) can be administered sequentially in any order, or simultaneously. In some aspects, the combination of the present disclosure is administered to a subject who is also receiving therapy with one or more other agents or methods. In other aspects, the combination is administered in conjunction with surgical treatments. The therapy regimen can be additive, or it can produce synergistic results.

### Pharmaceutical Compositions

The disclosure provides pharmaceutical compositions comprising the combination of a STING agonist molecule with an IL-15/IL-15Ra complex formulated together or separately with a pharmaceutically acceptable carrier. The STING agonist molecule and the IL-15/IL-15Ra complex can be administered to a patient as a “non-fixed combination” meaning that the STING agonist molecule and IL-15/IL-15Ra complex are administered as separate entities either simultaneously, concurrently or sequentially with no specific time limits, wherein such administration provides therapeutically effective levels of the two agents in the body of the patient. The term “non-fixed combination” thus defines especially a “kit of parts” in the sense that the combination agents (i) a STING agonist molecule and (ii) an IL-15/IL-15Ra complex as defined herein can be dosed independently of each other or by use of different fixed combinations with distinguished amounts of the combination agents, i.e. simultaneously or at different time points, where the combination agents can also be used as entirely separate pharmaceutical dosage forms or pharmaceutical formulations that are also sold independently of each other and where instructions for the possibility of their combined use is or are provided in the package equipment, e.g. leaflet or the like, or in other information e.g. provided to physicians and medical staff. The independent formulations or the parts of the kit of parts can then, e.g. be administered simultaneously or chronologically staggered, that is at different time points and with equal or different time intervals for any part of the kit of parts. In a specific embodiment, the time intervals are chosen such that the effect on the treated disease in the combined use of the parts is larger than the effect which would be obtained by use of only any one of the combination agents (i) and (ii), thus being jointly active. The ratio of the total amounts of the combination agent (i) to the combination agent (ii) to be administered in the combined preparation can be varied, e.g. in order to cope with the needs of a patient sub-population to be treated or the needs of the single patient which different needs can be due to age, sex, body weight, etc. of the patients.

A pharmaceutical composition of the present disclosure can additionally contain one or more other therapeutic agents that are suitable for treating sarcomas, adenocarcinomas, blastomas, and carcinomas, of the various organ systems, such as those affecting liver, lung, breast, lymphoid, biliarintestinal (e.g., colon), genitourinary tract (e.g., renal, urothelial cells), prostate and pharynx. Adenocarcinomas include malignancies such as most colon cancers, rectal cancer, renal-cell carcinoma, liver cancer, small cell lung cancer, non-small cell carcinoma of the lung, cancer of the small intestine and cancer of the esophagus. In one embodiment, the cancer is a melanoma, e.g., an advanced stage melanoma. Examples of other cancers that can be treated include bone cancer, pancreatic cancer, skin cancer, cancer of the head or neck, cutaneous or intraocular malignant melanoma, uterine cancer, ovarian cancer, rectal cancer, colorectal cancer, cancer of the peritoneum, stomach or gastric cancer, esophageal cancer, salivary gland carcinoma, testicular cancer, uterine cancer, carcinoma of the fallopian tubes, carcinoma of the endometrium, carcinoma of the cervix, carcinoma of the vagina, carcinoma of the vulva, penile carcinoma, glioblastoma, neuroblastoma, cervical cancer, Hodgkin Disease, non-Hodgkin lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, neuroendocrine tumors (including carcinoid tumors, gastrinoma, and islet cell cancer), mesothelioma, schwannoma (including acoustic neuroma), meningioma, epidermoid cancer, squamous cell cancer, T-cell lymphoma, B-cell acute lymphoid leukemia (“BALL”), T-cell acute lymphoid leukemia (“TALL”), acute lymphoid leukemia (ALL); one or more chronic leukemias including but not limited to, e.g., chronic myelogenous leukemia (CML), chronic lymphocytic leukemia (CLL); additional hematologic cancers or hematologic conditions including, but not limited to, e.g., B cell prolymphocytic leukemia, blastic plasmacytoid dendritic cell neoplasm, Burkitt's lymphoma, diffuse large B cell lymphoma, Follicular lymphoma, Hairy cell leukemia, small cell- or a large cell-follicular lymphoma, malignant lymphoproliferative conditions, MALT lymphoma, mantle cell lymphoma, Marginal zone lymphoma, multiple myeloma, myelodysplasia and myelodysplastic syndrome, non-Hodgkin lymphoma, plasmablastic lymphoma and plasmacytoid dendritic cell neoplasm.

A pharmaceutical composition of the present disclosure can be administered with a pharmaceutically acceptable carrier to enhance or stabilize the composition, or facilitate preparation of the composition. Pharmaceutically acceptable carriers include solvents, dispersion media, coatings, antibacterial and antifungal agents, isotonic and absorption delaying agents, and the like that are physiologically compatible.

A pharmaceutical composition of the present disclosure can be administered by a variety of methods known in the art. The route and/or mode of administration can vary depending upon the desired results. Administration can be intravenous, intramuscular, intraperitoneal, or subcutaneous, or administered proximal to the site of the target. The pharmaceutically acceptable carrier should be suitable for intravenous, intramuscular, subcutaneous, parenteral, spinal or epidermal administration (e.g., by injection or infusion). Depending on the route of administration, the active compound, i.e., STING agonist molecule and/or IL-15/IL-15Ra complex, can be coated in a material to protect the compound from the action of acids and other natural conditions that can inactivate the compound.

The composition should be sterile and fluid. Fluidity can be maintained, for example, by use of coating such as lecithin, by maintenance of required particle size in the case of dispersion and by use of surfactants. In many cases, it is preferable to include isotonic agents, for example, sugars, polyalcohols such as mannitol or sorbitol, and sodium chloride in the composition. Long-term absorption of the injectable compositions can be brought about by including in the composition an agent which delays absorption, for example, aluminum monostearate or gelatin.

Pharmaceutical compositions of the disclosure can be prepared in accordance with methods well known and routinely practiced in the art. See, e.g., Remington: The Science and Practice of Pharmacy, Mack Publishing Co., 20th ed., (2000); and Sustained and Controlled Release Drug Delivery Systems, J. R. Robinson, ed., Marcel Dekker, Inc., New York, (1978). Pharmaceutical compositions are preferably manufactured under GMP conditions. Typically, a therapeutically effective dose or efficacious dose of the STING agonist molecule and the IL-15/IL-15Ra complex, of the combination, can be employed in pharmaceutical compositions. The STING agonist molecule and IL-15/IL-15Ra complex are formulated into pharmaceutically acceptable dosage forms by conventional methods known to those of skill in the art. Dosage regimens are adjusted to provide the optimum desired response (e.g., a therapeutic response). For example, for each component of the combination, a single bolus can be administered, several divided doses can be administered over time or the dose can be proportionally reduced or increased as indicated by the requirements of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

Actual dosage levels of the active ingredients in the pharmaceutical compositions of the present disclosure can be varied so as to obtain an amount of the active ingredient which is effective to achieve the desired therapeutic response for a particular patient, composition, and mode of administration, without being toxic to the patient. The selected dosage level depends upon a variety of pharmacokinetic factors including the activity of the particular components of the combination employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors.

A physician can start doses of the combination in a pharmaceutical composition at levels lower than that required to achieve the desired therapeutic effect and gradually increase the dosage until the desired effect is achieved. In general, effective doses of the compositions of the present disclosure, for the treatment of an disorder described herein vary depending upon many different factors, including means of administration, target site, physiological state of the patient, other medications administered, and whether treatment is prophylactic or therapeutic. Treatment dosages need to be titrated to optimize safety and efficacy. For administration of a STING agonist molecule, the dosage ranges from about 0.0001 to 100 mg/kg, and more usually 0.01 to 15 mg/kg, of the host body weight. An exemplary treatment regime entails systemic administration once every two weeks or once a month or once every 3 to 6 months. For subcutaneous administration of the IL-15/IL-15Ra complex, the dose ranges from about 0.25 to 8 μg/kg/day. An exemplary treatment regime entails subcutaneous administration in a treatment cycle of three times a week for two weeks, followed by a two week break before a repeat of the treatment cycle.

For a combination comprising a STING agonist molecule with an IL-15/IL-15Ra complex, the STING agonist molecule and/or IL-15/IL-15Ra complex can be administered on multiple occasions. Intervals between single dosages can be weekly, monthly or yearly. Intervals can also be irregular as indicated by measuring CD8 + T cells in the patient. Alternatively, the components of the combination can be administered as a sustained release formulation, in which case less frequent administration is required. Dosage and frequency vary depending on the half-life of the IL-15/IL-15Ra complex in the patient. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can be administered a prophylactic regime.

### Specific Embodiments, Citation and References

Various references, including patent applications, patents, and scientific publications, are cited herein; the disclosure of each such reference is hereby incorporated herein by reference in its entirety.

### EXAMPLES

The following examples are provided to illustrate but not to limit the scope of the discovery. Other variants of the discovery will be readily apparent to one of ordinary skill in the art and are encompassed by the appended claims.

### Example 1

### Combination of hetIL15 and STING100 in the MC38 Tumor Model

NK cells and CD8+ T cells are stimulated by IL-15/soluble IL-15Ra complexes (“hetIL-15”). STING100 stimulates the innate immune system, and thus these two immune activating agents can synergize and drive a robust anti-tumor immune response by: 1) increasing immune infiltration into the tumor, and 2) enhancing activation of the immune components infiltrating the tumor. To demonstrate this effect, an in vivo combination experiment in the MC38 tumor model was performed as detailed below.

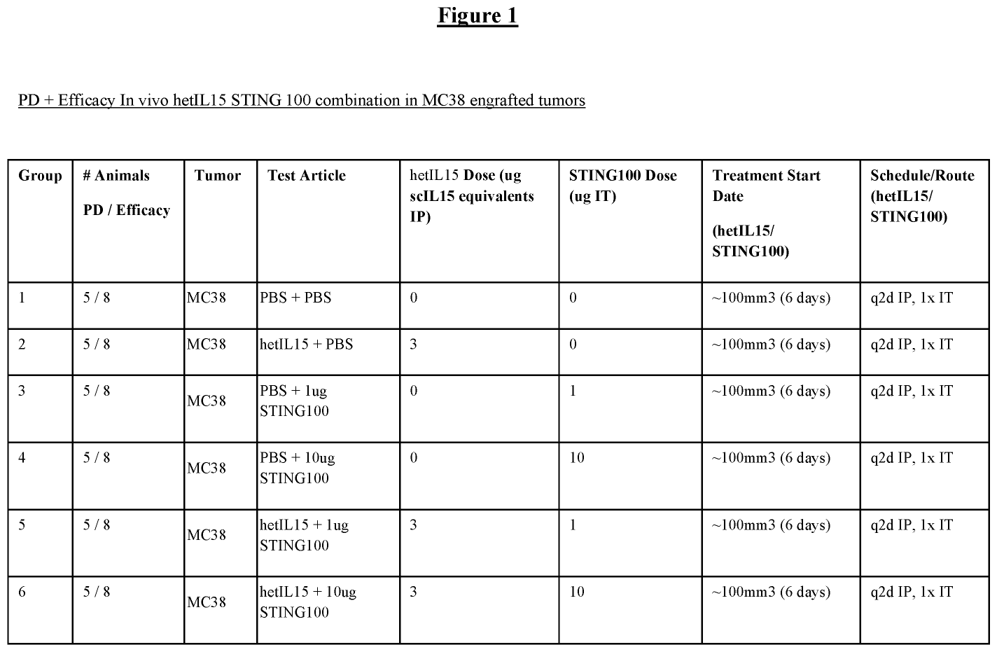

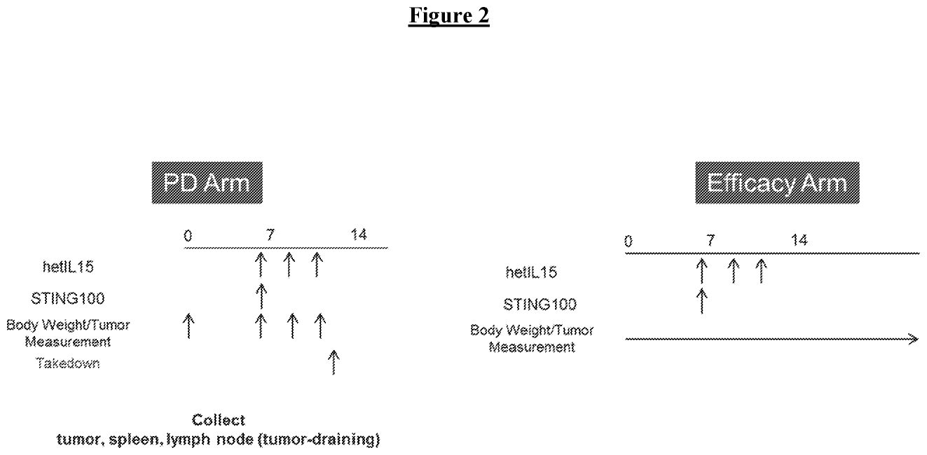

MC38 murine colon carcinoma cells (NCI, Rockville Md.) were grown in DMEM +10% Heat-Inactivated Fetal Bovine Serum. 6-8 week old C57B1/6 mice were purchased from Jackson Laboratories (Bar Harbor, Me.). Prior to implant, MC38 cells were washed once in PBS and resuspended at 1×10⁶cells/100 μL PBS and 1×10⁶ cells were implanted subcutaneously into the upper right flank. Tumors were allowed to grow for 6 days, and randomized into treatment groups with a mean tumor size of roughly 109 mm³ On Day 6 post-tumor implant, STING100 was injected into the tumor at a dose of either 1 μg or 10 μg in PBS with a volume of 50 μL, or PBS was injected as a control in the same volume. On days 6, 8, and 10 post-tumor implant, hetIL15 was injected at 3 μg (single-chain IL-15 equivalents) into the peritoneal cavity. The dosing amount of both molecules is shown in FIG. 1, with the timing of the dosing shown in FIG. 2. Eight (8) pre-assigned mice from each group were then measured by caliper for the duration of the study, until tumors reached a maximum size of 1,500 mm³.

On day 11 post-tumor implant, 5 pre-assigned mice from each group were euthanized and spleens, draining lymph node (axillary), non-draining lymph node (contralateral inguinal), and tumor were isolated. Single cell suspensions of spleens and lymph nodes were generated by passing the organs through 70 μm filters into PBS +2% Fetal Bovine Serum +2 mM EDTA. Tumors were digested in a solution of collagenase, dispase, and DNAse for approximately 3 rounds of 20 minutes, along with mechanical disruption between each round. Cells were then stained with an antibody panel, run on a BD Fortessa® (Becton-Dickinson, Franklin Lakes, N.J.) and analyzed in FlowJo® for immune presence and activation.

For mice from the initial challenge with no detectable tumor after 50 days, rechallenge was performed by injection of 1×10⁶ MC38 into the contralateral upper flank of the mice along with a set of previously unchallenged (naive) control age-matched mice, and monitoring of mice was again performed by caliper. Twenty-one (21) days after rechallenge, splenocytes were isolated from mice as described above, and were cultured for 48 hours in DMEM +10% FBS with either 1) alone, 2) at a 10:1 ratio with irradiated (10,000 rads) MC38, or 3) at a 1:2 ratio with anti-CD3/28 Dynabeads (Gibco). IFN-y production was then measured using an ELISA (R&D #DY485).

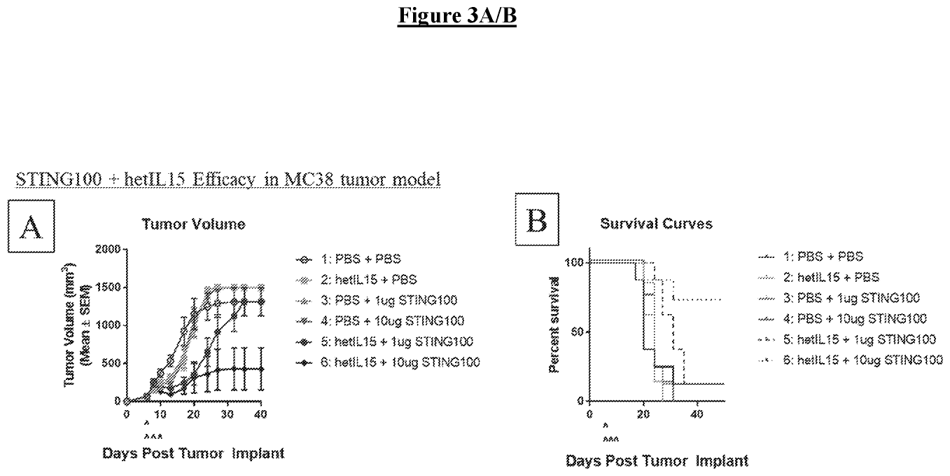

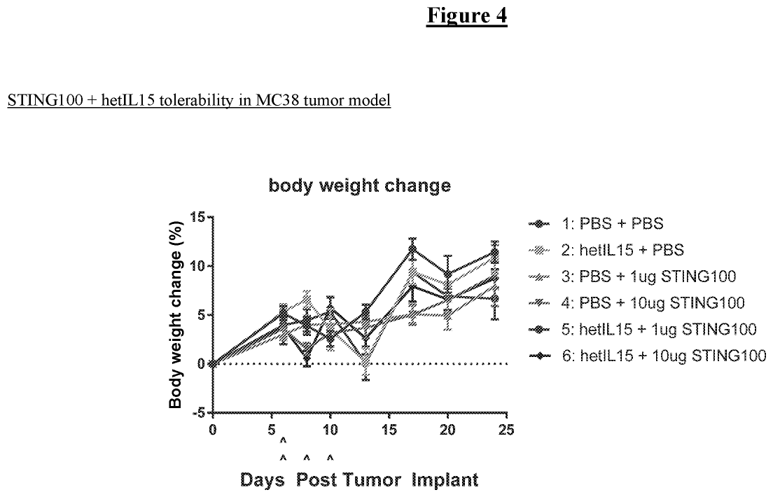

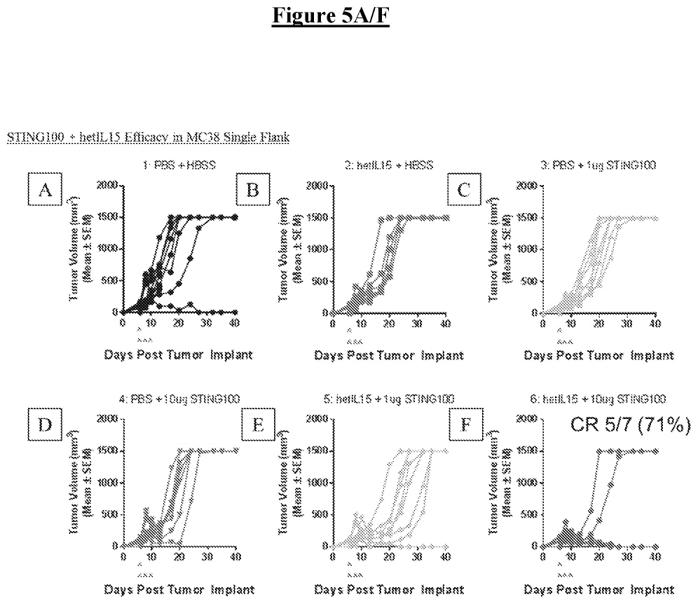

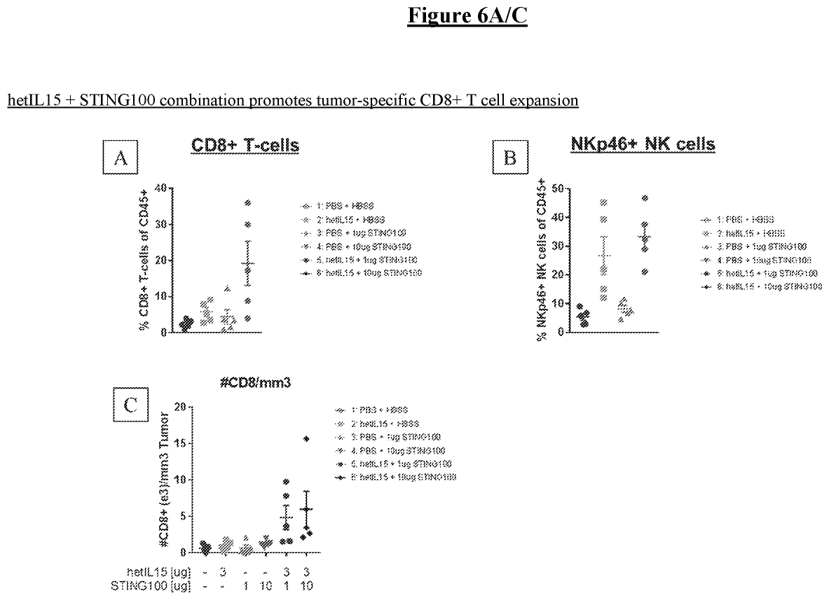

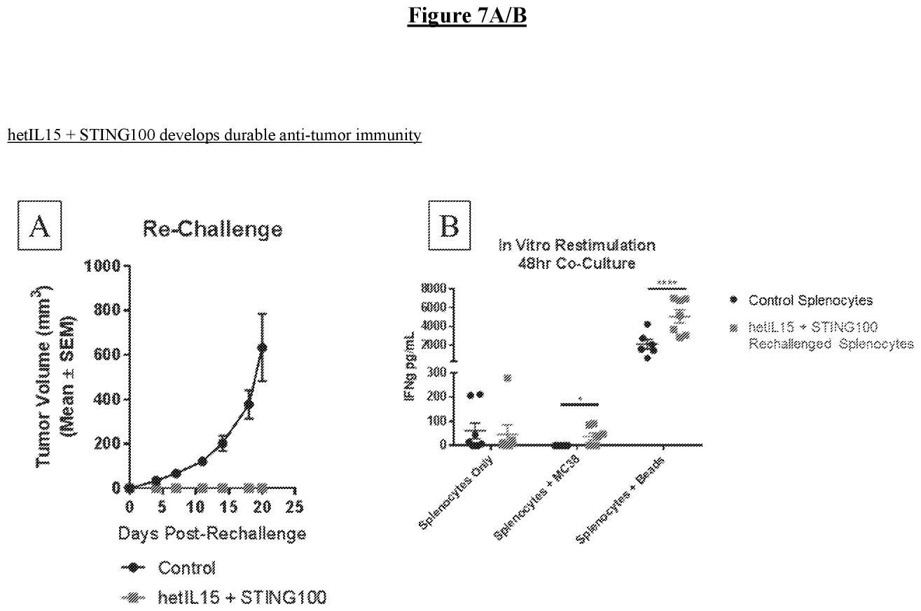

A strong synergistic effect was observed between hetIL15 and STING100, including an improved survival to initial challenge (FIG. 3A/B). The administration of the hetIL15/STING100 combination was well tolerated, with no change in body weight (FIG. 4). The results of each individual mouse cohort is broken out in FIG. 5A/F, including the complete response and eradication of the tumor in 71% (5/7) of mice as shown in FIG. 5F. Robust immune infiltrate and activation, especially for tumor specific CD8+ T cells and NK cells (FIG. 6A/C) was seen. Lastly, a strong response to rechallenge both in vivo and in vitro (FIG. 7A/B) was observed, indicating the hetIL15/STING100 combination generated durable anti-tumor immunity.

TABLE 1
Sequence Table

| | | |
|---|---|---|
| SEQ ID | | |
| NO: | Description | Sequence |

IL-15 & IL-15Ra Related Sequences

| | | |
|---|---|---|
| 1 | IL-15 (with | MRISKPHLRSISIQCYLCLLLNSHFITEAGIHVFILGCFSAGLPKTEANWNVV |
| | signal | ISDLKKIEDLIQSMHIDATLYTESDVHPSCKVTAMKCFLLELQVISLESGDAS |
| | peptide) aa | IHDTVENLIILANNSLSSNGNVTESGCKECEELEEKNIKEFLQSFVHIVQMFI |
| | human | NTS |
| 2 | Coding | atgagaattt cgaaaccaca tttgagaagt atttccatcc |
| | sequence of | agtgctactt gtgtttactt ctaaacagtc attttctaac |
| | immature/ | tgaagctggc attcatgtct tcattttggg ctgtttcagt |
| | precursor | gcagggcttc ctaaaacaga agccaactgg gtgaatgtaa |
| | form of | taagtgattt gaaaaaaatt gaagatctta ttcaatctat |
| | human IL-15 | gcatattgat gctactttat atacggaaag tgatgttcac |
| | including | cccagttgca aagtaacagc aatgaagtgc tttctcttgg |
| | signal | agttacaagt tatttcactt gagtccggag atgcaagtat |
| | peptide DNA | tcatgataca gtagaaaatc tgatcatcct agcaaacaac |
| | | agtttgtctt ctaatgggaa tgtaacagaa tctggatgca |
| | | aagaatgtga ggaactggag gaaaaaaata ttaaagaatt |
| | | tttgcagagt tttgtacata ttgtccaaat gttcatcaac |
| | | acttcttga |
| 3 | IL-15 | atgtggctcc agagcctgct actcctgggg acggtggcct |
| | expression | gcagcatctc gaactgggtg aacgtgatct cggacctgaa |
| | construct | gaagatcgag gacctcatcc agtcgatgca catcgacgcg |
| | DNA (human | acgctgtaca cggagtcgga cgtccacccg tcgtgcaagg |
| | IL-15 with | tcacggcgat gaagtgcttc ctcctggagc tccaagtcat |
| | GMCSF | ctcgctcgag tcgggggacg cgtcgatcca cgacacggtg |
| | signal | gagaacctga tcatcctggc gaacaactcg ctgtcgtcga |
| | peptide) | acgggaacgt cacggagtcg ggctgcaagg agtgcgagga |
| | | gctggaggag aagaacatca aggagttcct gcagtcgttc |
| | | gtgcacatcg tccagatgtt catcaacacg tcgtga |
| 4 | IL-15 codon | atgcggatctcgaagccgcacctgcggtcgatatcgatccagtgctacctgtg |
| | optimized | cctgctcctgaactcgcacttcctcacggaggccggtatacacgtcttcatcc |
| | DNA | tgggctgcttctcggcggggctgccgaagacggaggcgaactgggtgaacgtg |
| | | atctcggacctgaagaagatcgaggacctcatccagtcgatgcacatcgacgc |
| | | gacgctgtacacggagtcggacgtccacccgtcgtgcaaggtcacggcgatga |
| | | agtgcttcctcctggagctccaagtcatctcgctcgagtcgggggacgcgtcg |
| | | atccacgacacggtggagaacctgatcatcctggcgaacaactcgctgtcgtc |
| | | gaacgggaacgtcacggagtcgggctgcaaggagtgcgaggagctggaggaga |
| | | agaacatcaaggagttcctgcagtcgttcgtgcacatcgtccagatgttcatc |
| | | aacacgtcgtga |
| 5 | IL-15 codon | MRISKPHLRSISIQCYLCLLLNSHFLTEAGIHVFILGCFSAGLPKTEANWVNV |
| | optimized | ISDLKKIEDLIQSMHIDATLYTESDVHPSCKVTAMKCFLLELQVISLESGDAS |
| | aa | IHDTVENLIILANNSLSSNGNVTESGCKECEELEEKNIKEFLQSFVHIVQMFI |
| | | NTS |
| 6 | IL-15Ra | MAPRRARGCRTLGLPALLLLLLLRPPATRGITCPPPMSVEHADIWVKSYSLYS |
| | (full | RERYICNSGFKRKAGTSSLTECVLNKATNVAHWTTPSLKCIRDPALVHQRPAP |
| | length | PSTVTTAGVTPQPESLSPSGKEPAASSPSSNNTAATTAAIVPGSQLMPSKSPS |
| | human) aa | TGTTEISSHESSHGTPSQTTAKNWELTASASHQPPGVYPQGHSDTTVAISTST |
| | with signal | VLLCGLSAVSLLACYLKSRQTPPLASVEMEAMEALPVTWGTSSRDEDLENCSH |
| | peptide | HL |
| 7 | IL-15Ra | MAPRRARGCRTLGLPALLLLLLLRPPATRGITCPPPMSVEHADIWVKSYSLYS |
| | (soluble | RERYICNSGFKRKAGTSSLTECVLNKATNVAHNTTPSLKCIRDPALVHQRPAP |
| | human PQG | PSTVTTAGVTPQPESLSPSGKEPAASSPSSNNTAATTAAIVPGSQLMPSKSPS |
| | termination | TGTTEISSHESSHGTPSQTTAKNWELTASASHQPPGVYPQG |
| | with signal | |
| | peptide) | |
| 8 | Coding | atggccccgc ggcgggcgcg cggctgccgg accctcggtc |
| | sequence of | tcccggcgct gctactgctg ctgctgctcc ggccgccggc |
| | full length | gacgcggggc atcacgtgcc ctccccccat gtccgtggaa |
| | human IL- | cacgcagaca tctgggtcaa gagctacagc ttgtactcca |
| | 15Ra DNA | gggagcggta catttgtaac tctggtttca agcgtaaagc |
| | | cggcacgtcc agcctgacgg agtgcgtgtt gaacaaggcc |
| | | acgaatgtcg cccactggac aacccccagt ctcaaatgca |
| | | ttagagaccc tgccctggtt |
| | | caccaaaggc cagcgccacc ctccacagta acgacggcag |
| | | gggtgacccc acagccagag agcctctccc cttctggaaa |
| | | agagcccgca gcttcatctc ccagctcaaa caacacagcg |
| | | gccacaacag cagctattgt cccgggctcc cagctgatgc |
| | | cttcaaaatc accttccaca ggaaccacag agataagcag |
| | | tcatgagtcc tcccacggca ccccctctca gacaacagcc |
| | | aagaactggg aactcacagc atccgcctcc caccagccgc |
| | | caggtgtgta tccacagggc cacagcgaca ccactgtggc |
| | | tatctccacg tccactgtcc tgctgtgtgg gctgagcgct |
| | | gtgtctctcc tggcatgcta cctcaagtca aggcaaactc |
| | | ccccgctggc cagcgttgaa atggaagcca tggaggctct |
| | | gccggtgact tgggggacca gcagcagaga tgaagacttg |
| | | gaaaactgct ctcaccacct atga |
| 9 | Coding | atggccccgc ggcgggcgcg cggctgccgg accctcggtc |
| | sequence of | tcccggcgct gctactgctg ctgctgctcc ggccgccggc |
| | immature | gacgcggggc atcacgtgcc ctccccccat gtccgtggaa |
| | form of the | cacgcagaca tctgggtcaa gagctacagc ttgtactcca |
| | soluble | gggagcggta catttgtaac tctggtttca agcgtaaagc |
| | human IL- | cggcacgtcc agcctgacgg agtgcgtgtt gaacaaggcc |
| | 15Ra DNA | acgaatgtcg cccactggac aacccccagt ctcaaatgca |
| | | ttagagaccc tgccctggtt caccaaaggc cagcgccacc |
| | | ctccacagta acgacggcag gggtgacccc acagccagag |
| | | agcctctccc cttctggaaa agagcccgca gcttcatctc |
| | | ccagctcaaa caacacagcg gccacaacag cagctattgt |
| | | cccgggctcc cagctgatgc cttcaaaatc accttccaca |
| | | ggaaccacag agataagcag tcatgagtcc tcccacggca |
| | | ccccctctca gacaacagcc aagaactggg aactcacagc |
| | | atccgcctcc caccagccgc caggtgtgta tccacagggc |
| 10 | IL-15Ra w/o | ITCPPPMSVEHADIWVKSYSLYSRERYICNSGFKRKAGTSSLTECVLNKATNV |
| | signal | AHWTTPSLKCIRDPALVHQRPAPPSTVTTAGVTPQPESLSPSGKEPAASSPSS |
| | peptide | NNTAATTAAIVPGSQLMPSKSPSTGTTEISSHESSHGTPSQTTAKNWELTASA |
| | with PQG | SHQPPGVYPQG |
| | termination | |
| 11 | IL-15Ra | atggccccgaggcgggcgcgaggctgccggaccctcggtctcccggcgctgct |
| | codon | actgctcctgctgctccggccgccggcgacgcggggcatcacgtgcccgcccc |
| | optimized | ccatgtccgtggagcacgcagacatctgggtcaagagctacagcttgtactcc |
| | DNA | cgggagcggtacatctgcaactcgggtttcaagcggaaggccggcacgtccag |
| | | cctgacggagtgcgtgttgaacaaggccacgaatgtcgcccactggacgaccc |
| | | cctcgctcaagtgcatccgcgacccggccctggttcaccagcggcccgcgcca |
| | | ccctccaccgtaacgacggcgggggtgaccccgcagccggagagcctctcccc |
| | | gtcgggaaaggagcccgccgcgtcgtcgcccagctcgaacaacacggcggcca |
| | | caactgcagcgatcgtcccgggctcccagctgatgccgtcgaagtcgccgtcc |
| | | acgggaaccacggagatcagcagtcatgagtcctcccacggcaccccctcgca |
| | | aacgacggccaagaactgggaactcacggcgtccgcctcccaccagccgccgg |
| | | gggtgtatccgcaaggccacagcgacaccacggtggcgatctccacgtccacg |
| | | gtcctgctgtgtgggctgagcgcggtgtcgctcctggcgtgctacctcaagtc |
| | | gaggcagactcccccgctggccagcgttgagatggaggccatggaggctctgc |
| | | cggtgacgtgggggaccagcagcagggatgaggacttggagaactgctcgcac |
| | | cacctataa |
| 12 | IL-15Ra | MAPRRARGCRTLGLPALLLLLLLRPPATRGITCPPPMSVEHADIWVKSYSLYS |
| | codon | RERYICNSGFKRKAGTSSLTECVLNKATNVAHWTTPSLKCIRDPALVHQRPAP |
| | optimized | PSTVTTAGVTPQPESLSPSGKEPAASSPSSNNTAATTAAIVPGSQLMPSKSPS |
| | aa | TGTTEISSHESSHGTPSQTTAKNWELTASASHQPPGVYPQGHSDTTVAISTST |
| | | VLLCGLSAVSLLACYLKSRQTPPLASVEMEAMEALPVTWGTSSRDEDLENCSH |
| | | HL |
| 13 | IL-15Ra | atggccccgaggcgggcgcgaggctgccggaccctcggtctcccggcgctgct |
| | codon | actgctcctgctgctccggccgccggcgacgcggggcatcacgtgcccgcccc |
| | optimized | ccatgtccgtggagcacgcagacatctgggtcaagagctacagcttgtactcc |
| | DNA | cgggagcggtacatctgcaactcgggtttcaagcggaaggccggcacgtccag |
| | | cctgacggagtgcgtgttgaacaaggccacgaatgtcgcccactggacgaccc |
| | | cctcgctcaagtgcatccgcgacccggccctggttcaccagcggcccgcgcca |
| | | ccctccaccgtaacgacggcgggggtgaccccgcagccggagagcctctcccc |
| | | gtcgggaaaggagcccgccgcgtcgtcgcccagctcgaacaacacggcggcca |
| | | caactgcagcgatcgtcccgggctcccagctgatgccgtcgaagtcgccgtcc |
| | | acgggaaccacggagatcagcagtcatgagtcctcccacggcaccccctcgca |
| | | aacgacggccaagaactgggaactcacggcgtccgcctcccaccagccgccgg |
| | | gggtgtatccgcaaggccacagcgacaccacgtaa |
| 14 | IL-15Ra | MAPRRARGCRTLGLPALLLLLLLRPPATRGITCPPPMSVEHADIWVKSYSLYS |
| | codon | RERYICNSGFKRKAGTSSLTECVLNKATNVAHWTTPSLKCIRDPALVHQRPAP |
| | optimized | PSTVTTAGVTPQPESLSPSGKEPAASSPSSNNTAATTAAIVPGSQLMPSKSPS |
| | aa | TGTTEISSHESSHGTPSQTTAKNWEITASASHQPPGVYPQGHSDTT |
| 15 | C-terminal | PQGHSDTT |
| | end of the | |
| | soluble | |
| | form of | |
| | human IL- | |
| | 15Ra | |
| 16 | C-terminal | PQGHSDT |
| | end of the | |
| | soluble | |
| | form of | |
| | human IL- | |
| | 15Ra | |
| 17 | C-terminal | PQGHSD |
| | end of the | |
| | soluble | |
| | form of | |
| | human IL- | |
| | 15Ra | |
| 18 | C-terminal | PQGHS |
| | end of the | |
| | soluble | |
| | form of | |
| | human IL- | |
| | 15Ra | |
| 19 | C-terminal | PQGH |
| | end of the | |
| | soluble | |
| | form of | |
| | human IL- | |
| | 15Ra | |
| 20 | C-terminal | PQG |
| | end of the | |
| | soluble | |
| | form of | |
| | human IL- | |
| | 15Ra | |
| 21 | Soluble | ITCPPPMSVEHADIWVKSYSLYSRERYICNSGFKRKAGTSSLTECVLNKATNV |
| | form of | AHWTTPSLKCIRDPALVHQRPAPPSTVTTAGVTPQPESLSPSGKEPAASSPSS |
| | human IL- | NNTAATTAAIVPGSQLMPSKSPSTGTTEISSHESSHGTPSQTTAKNWEITASA |
| | 15Ra | SHQPPGVYPQGHSDTT |
| 22 | IL-15Ra | NWELTASASHQPPGVYPQG |
| | O-glycosylation | |
| | on Thr5 | |
| 23 | IL-15Ra N- | ITCPPPMSVEHADIWVK |
| | glycosylation | |
| | on Ser 8 | |
| 24 | IL-15Ra N- | ITCPPPMSVEHADIWVKSYSLYSRERYICNS |
| | glycosylation | |
| | on Ser | |
| | 18, 20, 23 | |
| | or 31 | |
| 25 | IL-15Ra | RXXR |
| | heterologous | |
| | protease | |
| | cleavage | |
| | site | |
| | recognized | |
| | by furin | |
| | protease | |
| | Arg-X-X-Arg | |
| | Xaa = any | |
| | amino acid | |
| 26 | IL-15Ra | XXPRXX |
| | heterologous | |
| | protease | |
| | cleavage | |
| | site A-B- | |
| | Pro-Arg-X-Y | |
| | 1, 2 Xaa = | |
| | hydrophobic | |
| | amino acids | |
| | 5, 6 Xaa = | |
| | nonacidic | |
| | amino acids | |
| 27 | IL-15RaFc | MAPRRARGCRTLGLPALLLLLLLRPPATRGITCPPPMSVEHADIWVKSYSLYS |
| | fusion | RERYICNSGFKRKAGTSSLTECVLNKATNVAHWTTPSLKCIRDPALVHQRPAP |
| | protein | PSTVTTAGVTPQPESLSPSGKEPAASSPSSNNTAATTAAIVPGSQLMPSKSPS |
| | | TGTTEISSHESSHGTPSQTTAKNWELTASASHQPPGVYPQGHSDTTPKSCDKT |
| | | HTCPPCPAPELLGGPSVFLFPPKPKDTLMISRTPEVTCVVVDVSHEDPEVKFN |
| | | WYVDGVEVHNAKTKPREEQYNSTYRVVSVLTVLHQDWLNGKEYKCKVSNKALP |
| | | APIEKTISKAKGQPREPQVYTLPPSRDELTKNQVSLTCLVKGFYPSDIAVEWE |
| | | SNGQPENNYKTTPPVLDSDGSFFLYSKLTVDKSRWQQGNVFSCSVMHEALHNH |
| | | YTQKSLSLSPGK |
| 28 | IL-15RaFc | MAPRRARGCRTLGLPALLLLLLLRPPATRGITCPPPMSVEHADIWVKSYSLYS |
| | fusion | RERYICNSGFKRKAGTSSLTECVLNKATNVAHWTTPSLKCIRDPALVHQRPAP |
| | prorein | PSTVTTAGVTPQPESLSPSGKEPAASSPSSNNTAATTAAIVPGSQLMPSKSPS |
| | | TGTTEISSHESSHGTPSQTTAKNWELTASASHQPPGVYPQGPKSCDKTHTCPP |
| | | CPAPELLGGPSVFLFPPKPKDTIMISRTPEVTCVVVDVSHEDPEVKFNWYVDG |
| | | VEVHNAKTKPREEQYNSTYRVVSVLTVLHQDWLNGKEYKCKVSNKALPAPIEK |
| | | TISKAKGQPREPQVYTLPPSRDELTKNQVSLTCLVKGFYPSDIAVEWESNGQP |
| | | ENNYKTTPPVLDSDGSFFLYSKLTVDKSRWQQGNVFSCSVMHEALHNHYTQKS |
| | | LSLSPGK |

1. A combination comprising:
i) a STING agonist molecule; and
ii) an interleukin-15 (IL-15)/IL-15 receptor alpha (IL-15Ra) complex.

2. The combination of claim 1, wherein the IL-15/IL-15Ra complex is a heterodimeric complex of human IL-15 and human soluble IL-15Ra.

3. The combination of claim 2, wherein the human IL-15 comprises residues 49 to 162 of the amino acid sequence of SEQ ID NO: 1 and the human soluble IL-15Ra comprises the amino acid sequence of SEQ ID NO: 10.

4. The combination of claim 1, wherein the STING agonist molecule is selected from the group consisting of STING100, STING101, STING102, STING103, STING104, STING105, STING106 and STING107.

5. The combination of claim 1, wherein the STING agonist molecule comprises STING100.

6. The combination of claim 1, wherein the IL-15/IL-15Ra complex is glycosylated.

7. The combination according to claim 1 for use as a medicament, wherein the STING agonist molecule and the IL-15/IL-15Ra complex are administered simultaneously or sequentially.

8. The combination according to claim 1 comprising the STING agonist molecule and IL-15/IL-15Ra complex in a therapeutically effective amount for the treatment cancer.

9. Use of a combination according to claim 1 for the manufacture of a medicament for the treatment of cancer.

10. The use according to claim 9, wherein the cancer includes colon cancers, liver cancer, small cell lung cancer, non-small cell carcinoma of the lung, melanoma, pancreatic cancer, skin cancer, uterine cancer, ovarian cancer, gastric cancer, Kaposi's sarcoma, and squamous cell cancer.

11. The use according to claim 9, wherein the cancer includes T-cell lymphoma, B-cell acute lymphoid leukemia (“BALL”), T-cell acute lymphoid leukemia (“TALL”), acute lymphoid leukemia (ALL); one or more chronic leukemias including but not limited to, e.g., chronic myelogenous leukemia (CML), chronic lymphocytic leukemia (CLL); Burkitt's lymphoma, diffuse large B cell lymphoma, Follicular lymphoma, Hairy cell leukemia, small cell- or a large cell-follicular lymphoma, malignant lymphoproliferative conditions, MALT lymphoma, mantle cell lymphoma, Marginal zone lymphoma, multiple myeloma, myelodysplasia and myelodysplastic syndrome, non-Hodgkin lymphoma, plasmablastic lymphoma and plasmacytoid dendritic cell neoplasm.

12. A method for the treatment of a cancer, the method comprising administering an effective amount of the combination of claim 1 to a subject in need thereof, wherein the cancer is resistant or refractory.

13. The method of claim 12, wherein the STING agonist molecule and the IL-15/IL-15Ra complex are administered simultaneously or sequentially.

14. The method of claim 12 further comprising administering an additional therapeutic agent.

15. A method of promoting tumor specific CD8+ T cell expansion, comprising administering an effective amount of an IL-15/IL-15Ra complex in combination STING agonist molecule.

16. The method of claim 15, wherein the STING agonist molecule is selected from the group consisting of STING100, STING101, STING102, STING103, STING104, STING105, STING106 and STING107.

17. The method of claim 16, wherein the STING agonist molecule comprises STING100.

18. The method of claim 15, wherein the IL-15/IL-15Ra complex is a heterodimeric complex of human IL-15 and human soluble IL-15Ra.

19. The method of claim 18, wherein the human IL-15 comprises residues 49 to 162 of the amino acid sequence of SEQ ID NO: 1 and the human soluble IL-15Ra comprises the amino acid sequence of SEQ ID NO: 10.

20. The method of claim 15 comprising administering the STING agonist molecule and IL-15/IL-15Ra complex simultaneously or sequentially.

21. The method according to claim 15, wherein the tumor specific CD8+ T cell expansion is therapeutically effective for the treatment of colon cancers, liver cancer, small cell lung cancer, non-small cell carcinoma of the lung, melanoma, pancreatic cancer, skin cancer, uterine cancer, ovarian cancer, gastric cancer, Kaposi's sarcoma, and squamous cell cancer.

22. The method according to claim 15, wherein the tumor specific CD8+ T cell expansion is therapeutically effective for the treatment of T-cell lymphoma, B-cell acute lymphoid leukemia (“BALL”), T-cell acute lymphoid leukemia (“TALL”), acute lymphoid leukemia (ALL); one or more chronic leukemias including but not limited to, e.g., chronic myelogenous leukemia (CML), chronic lymphocytic leukemia (CLL); Burkitt's lymphoma, diffuse large B cell lymphoma, Follicular lymphoma, Hairy cell leukemia, small cell- or a large cell-follicular lymphoma, malignant lymphoproliferative conditions, MALT lymphoma, mantle cell lymphoma, Marginal zone lymphoma, multiple myeloma, myelodysplasia and myelodysplastic syndrome, non-Hodgkin lymphoma, plasmablastic lymphoma and plasmacytoid dendritic cell neoplasm.

23. A method of promoting tumor specific NK cell expansion, comprising administering an effective amount of an IL-15/IL-15Ra complex in combination STING agonist molecule.

24. The method of claim 23, wherein the STING agonist molecule is selected from the group consisting of STING100, STING101, STING102, STING103, STING104, STING105, STING106 and STING107.

25. The method of claim 24, wherein the STING agonist molecule comprises STING100.

26. The method of claim 23, wherein the IL-15/IL-15Ra complex is a heterodimeric complex of human IL-15 and human soluble IL-15Ra.

27. The method of claim 26, wherein the human IL-15 comprises residues 49 to 162 of the amino acid sequence of SEQ ID NO: 1 and the human soluble IL-15Ra comprises the amino acid sequence of SEQ ID NO: 10.

28. The method of claim 23 comprising administering the STING agonist molecule and IL-15/IL-15Ra complex simultaneously or sequentially.

29. The method according to claim 23, wherein the tumor specific NK cell expansion is therapeutically effective for the treatment of colon cancers, liver cancer, small cell lung cancer, non-small cell carcinoma of the lung, melanoma, pancreatic cancer, skin cancer, uterine cancer, ovarian cancer, gastric cancer, Kaposi's sarcoma, and squamous cell cancer.

30. The method according to claim 23, wherein the tumor specific NK cell expansion is therapeutically effective for the treatment of T-cell lymphoma, B-cell acute lymphoid leukemia (“BALL”), T-cell acute lymphoid leukemia (“TALL”), acute lymphoid leukemia (ALL); one or more chronic leukemias including but not limited to, e.g., chronic myelogenous leukemia (CML), chronic lymphocytic leukemia (CLL); Burkitt's lymphoma, diffuse large B cell lymphoma, Follicular lymphoma, Hairy cell leukemia, small cell- or a large cell-follicular lymphoma, malignant lymphoproliferative conditions, MALT lymphoma, mantle cell lymphoma, Marginal zone lymphoma, multiple myeloma, myelodysplasia and myelodysplastic syndrome, non-Hodgkin lymphoma, plasmablastic lymphoma and plasmacytoid dendritic cell neoplasm.


SEQUENCE LISTING (28 sequences)

SEQ ID NO: 1 - protein, 162 aa, Homo sapiens
  IL-15 (with signal peptide) aa human
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 2 - DNA, 489 bp, Homo sapiens
  Coding sequence of immature/precursor form of human IL-15 including signal peptide DNA
atgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttga

SEQ ID NO: 3 - DNA, 396 bp, Artificial Sequence
  Description of Artificial Sequence Synthetic polynucleotide
  IL-15 expression construct DNA (human IL-15 with GMCSF signal peptide)
atgtggctccagagcctgctactcctggggacggtggcctgcagcatctcgaactgggtgaacgtgatctcggacctgaagaagatcgaggacctcatccagtcgatgcacatcgacgcgacgctgtacacggagtcggacgtccacccgtcgtgcaaggtcacggcgatgaagtgcttcctcctggagctccaagtcatctcgctcgagtcgggggacgcgtcgatccacgacacggtggagaacctgatcatcctggcgaacaactcgctgtcgtcgaacgggaacgtcacggagtcgggctgcaaggagtgcgaggagctggaggagaagaacatcaaggagttcctgcagtcgttcgtgcacatcgtccagatgttcatcaacacgtcgtga

SEQ ID NO: 4 - DNA, 489 bp, Artificial Sequence
  Description of Artificial Sequence Synthetic polynucleotide
  IL-15 codon optimized DNA
atgcggatctcgaagccgcacctgcggtcgatatcgatccagtgctacctgtgcctgctcctgaactcgcacttcctcacggaggccggtatacacgtcttcatcctgggctgcttctcggcggggctgccgaagacggaggcgaactgggtgaacgtgatctcggacctgaagaagatcgaggacctcatccagtcgatgcacatcgacgcgacgctgtacacggagtcggacgtccacccgtcgtgcaaggtcacggcgatgaagtgcttcctcctggagctccaagtcatctcgctcgagtcgggggacgcgtcgatccacgacacggtggagaacctgatcatcctggcgaacaactcgctgtcgtcgaacgggaacgtcacggagtcgggctgcaaggagtgcgaggagctggaggagaagaacatcaaggagttcctgcagtcgttcgtgcacatcgtccagatgttcatcaacacgtcgtga

SEQ ID NO: 5 - protein, 162 aa, Artificial Sequence; identical to SEQ ID NO: 1
  Description of Artificial Sequence Synthetic polypeptide
  IL-15 codon optimized aa

SEQ ID NO: 6 - protein, 267 aa, Artificial Sequence
  Description of Artificial Sequence Synthetic polypeptide
  IL-15Ra (full length human) aa with signal peptide
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 7 - protein, 200 aa, Artificial Sequence
  Description of Artificial Sequence Synthetic polypeptide
  IL-15Ra (soluble human PQG termination with signal peptide)
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQG

SEQ ID NO: 8 - DNA, 804 bp, Homo sapiens
  Coding sequence of full length human IL-15Ra DNA
atggccccgcggcgggcgcgcggctgccggaccctcggtctcccggcgctgctactgctgctgctgctccggccgccggcgacgcggggcatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccaccctccacagtaacgacggcaggggtgaccccacagccagagagcctctccccttctggaaaagagcccgcagcttcatctcccagctcaaacaacacagcggccacaacagcagctattgtcccgggctcccagctgatgccttcaaaatcaccttccacaggaaccacagagataagcagtcatgagtcctcccacggcaccccctctcagacaacagccaagaactgggaactcacagcatccgcctcccaccagccgccaggtgtgtatccacagggccacagcgacaccactgtggctatctccacgtccactgtcctgctgtgtgggctgagcgctgtgtctctcctggcatgctacctcaagtcaaggcaaactcccccgctggccagcgttgaaatggaagccatggaggctctgccggtgacttgggggaccagcagcagagatgaagacttggaaaactgctctcaccacctatga

SEQ ID NO: 9 - DNA, 600 bp, Homo sapiens
  Coding sequence of immature form of the soluble human IL-15Ra DNA
atggccccgcggcgggcgcgcggctgccggaccctcggtctcccggcgctgctactgctgctgctgctccggccgccggcgacgcggggcatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccaccctccacagtaacgacggcaggggtgaccccacagccagagagcctctccccttctggaaaagagcccgcagcttcatctcccagctcaaacaacacagcggccacaacagcagctattgtcccgggctcccagctgatgccttcaaaatcaccttccacaggaaccacagagataagcagtcatgagtcctcccacggcaccccctctcagacaacagccaagaactgggaactcacagcatccgcctcccaccagccgccaggtgtgtatccacagggc

SEQ ID NO: 10 - protein, 170 aa, Artificial Sequence
  Description of Artificial Sequence Synthetic polypeptide
  IL-15Ra w/o signal peptide with PQG termination
ITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQG

SEQ ID NO: 11 - DNA, 804 bp, Artificial Sequence
  Description of Artificial Sequence Synthetic polynucleotide
  IL-15Ra codon optimized DNA
atggccccgaggcgggcgcgaggctgccggaccctcggtctcccggcgctgctactgctcctgctgctccggccgccggcgacgcggggcatcacgtgcccgccccccatgtccgtggagcacgcagacatctgggtcaagagctacagcttgtactcccgggagcggtacatctgcaactcgggtttcaagcggaaggccggcacgtccagcctgacggagtgcgtgttgaacaaggccacgaatgtcgcccactggacgaccccctcgctcaagtgcatccgcgacccggccctggttcaccagcggcccgcgccaccctccaccgtaacgacggcgggggtgaccccgcagccggagagcctctccccgtcgggaaaggagcccgccgcgtcgtcgcccagctcgaacaacacggcggccacaactgcagcgatcgtcccgggctcccagctgatgccgtcgaagtcgccgtccacgggaaccacggagatcagcagtcatgagtcctcccacggcaccccctcgcaaacgacggccaagaactgggaactcacggcgtccgcctcccaccagccgccgggggtgtatccgcaaggccacagcgacaccacggtggcgatctccacgtccacggtcctgctgtgtgggctgagcgcggtgtcgctcctggcgtgctacctcaagtcgaggcagactcccccgctggccagcgttgagatggaggccatggaggctctgccggtgacgtgggggaccagcagcagggatgaggacttggagaactgctcgcaccacctataa

SEQ ID NO: 12 - protein, 267 aa, Artificial Sequence; identical to SEQ ID NO: 6
  Description of Artificial Sequence Synthetic polypeptide
  IL-15Ra codon optimized aa

SEQ ID NO: 13 - DNA, 618 bp, Artificial Sequence
  Description of Artificial Sequence Synthetic polynucleotide
  IL-15Ra codon optimized DNA
atggccccgaggcgggcgcgaggctgccggaccctcggtctcccggcgctgctactgctcctgctgctccggccgccggcgacgcggggcatcacgtgcccgccccccatgtccgtggagcacgcagacatctgggtcaagagctacagcttgtactcccgggagcggtacatctgcaactcgggtttcaagcggaaggccggcacgtccagcctgacggagtgcgtgttgaacaaggccacgaatgtcgcccactggacgaccccctcgctcaagtgcatccgcgacccggccctggttcaccagcggcccgcgccaccctccaccgtaacgacggcgggggtgaccccgcagccggagagcctctccccgtcgggaaaggagcccgccgcgtcgtcgcccagctcgaacaacacggcggccacaactgcagcgatcgtcccgggctcccagctgatgccgtcgaagtcgccgtccacgggaaccacggagatcagcagtcatgagtcctcccacggcaccccctcgcaaacgacggccaagaactgggaactcacggcgtccgcctcccaccagccgccgggggtgtatccgcaaggccacagcgacaccacgtaa

SEQ ID NO: 14 - protein, 205 aa, Artificial Sequence
  Description of Artificial Sequence Synthetic polypeptide
  IL-15Ra codon optimized aa
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTT

SEQ ID NO: 15 - protein, 8 aa, Artificial Sequence
  Description of Artificial Sequence Synthetic peptide
  C-terminal end of the soluble form of human IL-15Ra
PQGHSDTT

SEQ ID NO: 16 - protein, 7 aa, Artificial Sequence
  Description of Artificial Sequence Synthetic peptide
  C-terminal end of the soluble form of human IL-15Ra
PQGHSDT

SEQ ID NO: 17 - protein, 6 aa, Artificial Sequence
  Description of Artificial Sequence Synthetic peptide
  C-terminal end of the soluble form of human IL-15Ra
PQGHSD

SEQ ID NO: 18 - protein, 5 aa, Artificial Sequence
  Description of Artificial Sequence Synthetic peptide
  C-terminal end of the soluble form of human IL-15Ra
PQGHS

SEQ ID NO: 19 - protein, 4 aa, Artificial Sequence
  Description of Artificial Sequence Synthetic peptide
  C-terminal end of the soluble form of human IL-15Ra
PQGH

SEQ ID NO: 20 - protein, 3 aa, Artificial Sequence
  Description of Artificial Sequence Synthetic peptide
  C-terminal end of the soluble form of human IL-15Ra
PQG

SEQ ID NO: 21 - protein, 175 aa, Homo sapiens
ITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTT

SEQ ID NO: 22 - protein, 19 aa, Artificial Sequence
  Description of Artificial Sequence Synthetic peptide
  IL-15Ra O-glycosylation on Thr5
NWELTASASHQPPGVYPQG

SEQ ID NO: 23 - protein, 17 aa, Artificial Sequence
  Description of Artificial Sequence Synthetic peptide
  IL-15Ra N-glycosylation on Ser 8
ITCPPPMSVEHADIWVK

SEQ ID NO: 24 - protein, 31 aa, Artificial Sequence
  Description of Artificial Sequence Synthetic polypeptide
  IL-15Ra N-glycosylation on Ser 18, 20, 23 or 31
ITCPPPMSVEHADIWVKSYSLYSRERYICNS

SEQ ID NO: 25 - protein, 4 aa, Artificial Sequence
  Description of Artificial Sequence Synthetic peptide
  IL-15Ra heterologous protease cleavage site recognized by furin protease
  misc_feature (2)..(3): Xaa can be any naturally occurring amino acid
RXXR

SEQ ID NO: 26 - protein, 6 aa, Artificial Sequence
  Description of Artificial Sequence Synthetic peptide
  Il-15Ra heterologous protease cleavage site A-B-Pro-Arg-X-Y
  misc_feature (1)..(2): Xaa can be a hydrophobic amino acid
  misc_feature (5)..(6): Xaa can be nonacidic amino acid
XXPRXX

SEQ ID NO: 27 - protein, 436 aa, Artificial Sequence
  Description of Artificial Sequence Synthetic polypeptide
  IL-15RaFc fusion protein
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 431 aa, Artificial Sequence
  Description of Artificial Sequence Synthetic polypeptide
  IL-15RaFc fusion protein
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK